(19) United States
(12) Patent Application Publication
(10) Pub. No.: US 2004/0072174 A1
(43) Pub. Date: Apr. 15, 2004

(54) Calpain protease 12

(76) Inventors: Thomas Boehm, Vorrstetten (DE); Neil T. Dear, Oxon (GB)

Correspondence Address: LAHIVE & COCKFIELD, LLP., 28 STATE STREET, BOSTON, MA, 02109, US

(21) Appl. No.: 10/312,373

(22) PCT Filed: Jun. 29, 2001
(86) PCT No.: PCT/EP01/07457

(30) Foreign Application Priority Data
Jun. 30, 2000 (DE) 100 31 932.7

(51) Int. Cl. C12Q 1/68; G01N 33/574; C07H 21/04; A61K 38/48; A61K 39/395; C12N 9/64

(52) U.S. Cl. 435/6; 435/69.1; 435/226; 435/320.1; 435/325; 424/146.1; 530/388.26; 435/7.23; 536/23.2; 424/94.64

(57) ABSTRACT

A calpain protease 12 (Capn12) which has an amino acid sequence comprising the amino acids 1-342 of SEQ ID NO: 1 or an amino acid sequence selected from SEQ ID NO: 1, SEQ ID NO: 2, SEQ ID NO: 3 or SEQ ID NO: 4, and also functional analogs thereof, nucleic acids coding therefor, recombinant vectors containing said coding sequences, microorganisms transfected therewith, processes for the recombinant preparation of Capn12, and various applications of Capn12 and nucleic acids coding therefor are described.

The invention relates to a novel calpain protease called calpain protease 12 and to functional analogs thereof (denoted Capn12 hereinafter), to nucleic acids coding therefor, to recombinant vectors comprising said coding sequences, to microorganisms transfected therewith, to processes for the recombinant preparation of Capn12, and to various applications of Capn12 and of nucleic acids coding therefor.

Calpains are a family of cytosolic cysteine proteases. The classical calpains are composed of an isoform-specific large subunit (80 kDa) and an invariable small subunit (30 kDa) called Capn4. The large subunit of classical calpains has a four-domain structure, including a domain having protease activity and a C-terminal, calmodulin-like domain which can bind calcium. Recently however, a plurality of atypical mammalian homologs of the large calpain subunit have been found, which lack the characteristics of the active site of a protease (Capn6; Dear et al., 1997) and/or have an alternative C-terminal domain which possibly does not bind calcium (Capn5, Capn6, Capn7, Capn8; Dear et al., 1997; Braun et al., 1999; Franz et al., 1999). A summary of the currently known members of the gene family of mammalian calpains is available on the internet (http://Ag.Arizona.Edu/calpains).

The physiological role of calpains is unclear. Calpains cleave numerous substrates (Carafoli and Molinari, 1998) and have been connected with a multiplicity of processes, including apoptosis (Wang, 2000), cell division (Mellgren, 1997), modulation of the interactions of the integrin cytoskeleton (Schoenwaelder et al., 1997) and synaptic plasticity (Chan and Mattson, 1999). In addition, they have been linked to numerous pathological states, such as Alzheimer's disease, cataract, demyelination, cardiac ischemia, inflammation and traumatic brain injury (reviews: Carafoli and Molinari, 1998; Sorimachi et al., 1997; Wang and Yuen, 1997). Mutations in the Capn3 gene are responsible for limb-girdle muscular dystrophy type 2A (Richard et al., 1995).

It is an object of the present invention to provide novel homologs of the gene family of the large calpain subunit, because of the multiple physiological and pathological functions of the calpains. This would make it possible, for example, to find or develop novel active substances or novel targets for active substances, which can be used in the diagnosis, therapy and/or prophylaxis of pathological states in which calpains and substrates thereof or substances acting thereupon are involved.

We have found that this object is achieved by providing a novel calpain protease, calpain protease 12 (Capn12), and functional equivalents thereof.

Capn12 has an amino acid sequence comprising the amino acids 1-342 of SEQ ID NO: 1. The invention also relates to functional equivalents of said part sequence.

Preferred variants thereof have an amino acid sequence selected from SEQ ID NO: 1, SEQ ID NO: 2, SEQ ID NO: 3 or SEQ ID NO: 4. SEQ ID NO: 1 represents the amino acid sequence of splice variant Capn12A, SEQ ID NO: 2 represents the amino acid sequence of splice variant Capn12B, SEQ ID NO: 3 represents the amino acid sequence of splice variant Capn12C and SEQ ID NO: 4 represents the amino acid sequence of Capn12 from clone 914413 of the mouse EST database. The amino acid sequences SEQ ID NO: 1 to 4 are identical to one another in the N-terminal segment of amino acids 1-342. The predicted protein corresponding to splice variant Capn12A has 720 amino acids and a molecular weight of 80.5 kDa.

The invention also relates to the functional equivalents of Capn12 and of the specifically disclosed amino acid sequences. Functional equivalents include amino acid sequences which can be derived from the specific sequences and in which, compared with said specific sequences, one or more amino acids have been substituted, deleted, inverted, or added with negligible influence on the cysteine protease activity and/or on at least one further characteristic feature of Capn12. Further characteristic features of Capn12 are described below. The invention also includes Capn12-characteristic part sequences or Capn12 fragments which may be prepared, for example, by proteolytic digestion, peptide synthesis or recombinant DNA technology. Said part sequences of fragments may be used, for example, for preparing monoclonal or polyclonal antibodies.

The specifically disclosed amino acid sequences represent amino acid sequences of Capn12 splice variants, which were determined from a mouse EST database. However, the invention also relates to all Capn12 homologs of eukaryotic species, i.e. of invertebrates and vertebrates, in particular of mammals, e.g. rats, cats, dogs, pigs, sheep, cattle, horses, monkeys and particularly preferably humans, and further naturally occurring variants. The invention also includes all development-specifically and organ-specifically or tissue-specifically expressed Capn12 forms and artificially generated homologs which have the predetermined structural and/or functional properties.

The Capn12 of the invention in particular has cysteine protease activity. Its amino acid sequence contains the amino acids Cys, His and Asn (in the splice variants Capn12A, B and C: Cys105, His259 and Asn283) which are characteristic for the active site of cysteine proteases and are essential for its function. Moreover, the splice variant Capn12A has a distinctly acidic region and a calmodulin-like region which presumably binds Ca²⁺.

In addition, the murine gene coding for the Capn12 of the invention is located on chromosome 7 between the markers D7Mit72 (10.4 cM) and D7Mit267 (11.0 cM).

In addition, the Capn12 of the invention is expressed in mice, for example, in the cortex of the hair follicle of the skin.

Furthermore, the Capn12 of the invention is expressed in anagen of the hair cycle.

The invention also relates to calpain proteins which have at least one Capn12 of the invention. Besides Capn12 as large subunit, such a calpain protein preferably has a Capn4 as small protein subunit. Moreover, additional protein subunits such as, for example, regulatory subunits or subunits which mediate the localization of the protein in defined cell compartments may be present.

The invention furthermore also includes polynucleotides coding for a Capn12 of the invention and functional equivalents thereof and polynucleotides hybridizable therewith or complementary thereto, which include single-stranded and double-stranded DNA and RNA sequences. Said polynucleotides can be detected when screening genomic or cDNA libraries and, where appropriate, multiplied out of said libraries using suitable primers by means of PCR and subsequently be isolated using suitable probes, for example. Another possibility is to transform suitable microorganisms with polynucleotides or vectors of the invention and to multiply and subsequently isolate the microorganisms and thus the polynucleotides. Moreover, polynucleotides of the invention may also be synthesized chemically.

The invention also relates to polynucleotides having a nucleic acid sequence selected from SEQ ID NO: 5, SEQ ID NO: 6, SEQ ID NO: 7 or SEQ ID NO: 8. SEQ ID NO: 5 represents the nucleic acid sequence of the cDNA of splice variant Capn12A, SEQ ID NO: 6 represents the nucleic acid sequence of the cDNA of Capn12B, SEQ ID NO:

7 represents the nucleic acid sequence of the cDNA of Capn12C and SEQ ID NO: 8 represents the genomic murine Capn12 nucleic acid sequence which comprises all exon and intron sequences. The predicted genomic murine Capn12 sequence includes 21 exons and a genomic segment of 13 116 base pairs.

Functional equivalents of polynucleotides of the invention include sequences derived due to the degeneracy of the genetic code and thus silent nucleotide substitutions (i.e. without alterations in the resulting amino acid sequence) and conservative nucleotide substitutions (i.e. the relevant amino acid is replaced by an amino acid of identical charge, size, polarity and/or solubility). Functional equivalents of polynucleotides of the invention thus have a sequence modified by nucleotide substitution, deletion, inversion or addition, but likewise code for a functionally equivalent Capn12 such as, for example, one having identical or comparable cysteine protease activity. In particular, polynucleotides suitable according to the invention include at least one of the part sequences which code for characteristic amino acid sequences of Capn12.

The invention also relates to the primer sequences SEQ ID NO: 9, SEQ ID NO: 10, SEQ ID NO: 11, SEQ ID NO: 12, SEQ ID NO: 15, SEQ ID NO: 16, SEQ ID NO: 17 and SEQ ID NO: 18, which can hybridize to polynucleotides of the invention or are complementary thereto and may be used, for example, for the amplification thereof by RT-PCR or PCR.

The property of being able to hybridize to polynucleotides means the ability of a polynucleotide or oligonucleotide to bind to a nearly complementary sequence under stringent conditions, while unspecific bindings between noncomplementary partners are suppressed under said conditions. For this purpose, the sequences have to be 70-100%, preferably 90-100%, complementary. The property of complementary sequences to be able to bind specifically to one another is utilized, for example, in the Northern blot or Southern blot technique or for primer binding in PCR or RT-PCR. Commonly, oligonucleotides of 30 base pairs or longer are employed for this purpose. Stringent conditions mean, for example, in the Northern blot technique the use of a washing solution at 50-70° C., preferably 60-65° C., for example 0.1×SSC buffer with 0.1% SDS (20×SSC: 3M NaCl, 0.3M Na citrate, pH 7.0) for eluting unspecifically hybridized cDNA probes or oligonucleotides. As mentioned above, only highly complementary nucleic acids remain bound to one another here.

The invention also relates to expression cassettes which include at least one inventive polynucleotide which is operatively linked to at least one regulatory nucleic acid sequence. Preferably, a promoter sequence is located 5′ upstream from the polynucleotide of the invention and, in this way, makes a controlled Capn12 expression possible. Particularly preferably, a terminator sequence and, where appropriate, further common regulatory elements, in each case operatively linked with the sequence encoding Capn12, are located 3′ downstream from the polynucleotide of the invention.

An operative linkage means the sequential arrangement of regulatory and coding sequences such as, for example, promoter, coding sequence, terminator and, where appropriate, further regulatory elements, such that each of the regulatory elements can fulfil its function before, during or after expression of the coding sequence according to the requirements. Examples of further operatively linkable sequences are targeting sequences, translation amplifiers, enhancers, polyadenylation signals and the like. Suitable regulatory elements also include selectable markers, amplification signals, origins of replication and the like.

In addition to the artificial regulatory sequences, it is possible for the natural regulatory sequence still to be present in front of the actual structural gene. This natural regulation can, where appropriate, be switched off by genetic modification and the expression of the genes be increased or decreased. However, the expression cassette may also have a simple structure, i.e. no additional regulatory signals are inserted in front of the structural gene, and the natural promoter with its regulation is not removed. Instead, it is possible, for example, to mutate the natural regulatory sequence in such a way that regulation no longer takes place and gene expression is enhanced or diminished. The nucleic acid sequences may be present in one or more copies in the expression cassette.

Examples of suitable promoters are: cos, tac, trp, tet, trp-tet, lpp, lac, lpp-lac, laciq, T7, T5, T3, gal, trc, ara, SP6, λ-PR or λ-PL promoter, which are advantageously used in Gram-negative bacteria and also the Gram-positive promoters amy and SPO2, the yeast promoters ADC1, MFα, AC, P-60, CYC1, GAPDH or the plant promoters CaMV/35S, SSU, OCS, lib4, usp, STLS1, B33, nos or the ubiquitin or phaseolin promoter. Particularly preferred is the use of inducible promoters such as, for example, light-inducible and in particular temperature-inducible promoters such as the PᵣPₗ promoter.

It is possible in principle to use all natural promoters with their regulatory sequences. Moreover, it is also possible and advantageous to use synthetic promoters.

Said regulatory sequences are intended to make specific expression of the nucleic acid sequences and protein expression possible. This may mean, for example, depending on the host organism, that the gene is expressed or overexpressed only after induction, or that it is immediately expressed or overexpressed. Expression by said regulatory elements may also take place tissue-, cell- or development-specifically, if the vector is introduced into a higher organism, such as an animal or a plant.

In this connection, the regulatory sequences or vectors may preferably have a positive influence on, and thus increase or decrease, the expression. Thus, enhancement of the regulatory elements may advantageously take place at the level of transcription by using strong transcription signals such as promoters and/or enhancers. However, it is also possible to enhance translation by, for example improving the stability of the mRNA. Enhancers mean, for example, DNA sequences which bring about increased expression via an improved interaction between RNA polymerase and DNA.

An expression cassette of the invention is prepared by fusion of a suitable promoter with a suitable polynucleotide encoding Capn12, and also a terminator signal or polyadenylation signal. For this purpose, customary recombination and cloning techniques are used, such as, for example, the insertion via restriction enzyme cleavage sites or as described, for example, in T. Maniatis, E. F. Fritsch and J. Sambrook, Molecular Cloning: A Laboratory Manual, Cold Spring Harbor Laboratory, Cold Spring Harbor, N.Y. (1989), and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The invention also relates to recombinant vectors for transforming eukaryotic or prokaryotic hosts carrying a polynucleotide of the invention or an expression cassette of the invention. The said vectors allow Capn12 expression in a suitable host organism. Vectors are well known to the skilled worker and can be found, for example, in “Cloning Vectors” (Pouwels P. H. et al., eds, Elsevier, Amsterdam-New York-Oxford, 1985). Vectors mean, in addition to plasmids, also all other vectors known to the skilled worker, such as, for example, phages, viruses such as SV40, CMV, baculovirus and adenovirus, transposons, IS elements, plasmids, cosmids, and linear or circular DNA. Said vectors may undergo autonomous replication in the host organism or chromosomal replication.

The invention also relates to microorganisms containing a vector of the invention or to those expressing Capn12 endogenously. Said microorganisms may be used for producing recombinant Capn12. Advantageously, the above-described recombinant expression cassettes of the invention are introduced into and expressed in a suitable host system as part of an expression vector. This entails preferably using cloning and transfection methods familiar to the skilled worker, such as, for example, coprecipitation, protoplast fusion, electroporation, retroviral transfection and the like, in order to bring about expression of said nucleic acids in the particular expression system. Suitable systems are described, for example, in Current Protocols in Molecular Biology, F. Ausubel et al., eds, Wiley Interscience, New York 1997 and in J. Sambrook, E. F. Fritsch and T. Maniatis, Molecular Cloning: A Laboratory Manual, Cold Spring Harbour Laboratory, Cold Spring Harbour, N.Y. (1980).

Suitable host organisms for transformation with vectors of the invention are in principle all organisms which make it possible to express the polynucleotides of the invention, their allelic variants, their functional equivalents or derivatives. Host organisms mean, for example, bacteria, fungi, yeasts, plant or animal cells. Preferred organisms are bacteria such as those of the genera Escherichia, such as, for example, Escherichia coli, Streptomyces, Bacillus or Pseudomonas, eukaryotic microorganisms in particular yeasts such as Saccharomyces cerevisiae, Aspergillus, higher eukaryotic cells from animals or plants, for example Sf9 or CHO cells. If desired, the gene product may also be expressed in transgenic organisms such as transgenic animals such as, in particular, mice, sheep, or transgenic plants. The transgenic organisms may also be so-called knockout animals or plants in which the corresponding endogenous gene has been switched off, such as, for example, by mutation or partial or complete deletion.

Successfully transformed organisms can be selected by marker genes which are likewise included in the vector or in the expression cassette. Examples of such marker genes are genes for antibiotics resistance and for enzymes which catalyze a color reaction which brings about staining of the transformed cell. Said cells can then be selected by means of automated cell sorting. Microorganisms which have been successfully transformed with a vector and which carry an appropriate antibiotics resistance gene (e.g. G418 or hygromycin) can be selected on media culture containing the appropriate antibiotics. Marker proteins presented on the cell surface can be used for selection by means of affinity chromatography.

The combination of the host organisms and the vectors appropriate for the organisms, such as plasmids, viruses or phages, such as, for example, plasmids with the RNA polymerase/promoter system, phages λ, μ or other temperate phages or transposons and/or other advantageous regulatory sequences forms an expression system. The term “expression system” means, for example, the combination of mammalian cells such as CHO cells, and vectors such as pcDNA3neo vector, which are suitable for mammalian cells.

As described above, the gene product can also be expressed advantageously in transgenic animals, e.g. mice, sheep, or transgenic plants. It is likewise possible to program cell-free translation systems with the RNA derived from the nucleic acid.

The invention also relates to processes for preparing a Capn12 of the invention, in which processes a Capn12-producing microorganism is cultured, Capn12-expression is, where appropriate, induced and Capn12 is isolated from the culture. Capn12 can in this way also be produced on the industrial scale, if so desired.

The microorganism may be cultured and fermented according to known processes. Bacteria may be multiplied, for example, in TB or LB medium at from 20 to 40° C. and from pH 6 to pH 9. Suitable culturing conditions are described in detail, for example in T. Maniatis, E. F. Fritsch and J. Sambrook, Molecular Cloning: A Laboratory Manual, Cold, Spring Harbor Laboratory, Cold Spring Harbor, N.Y. (1989).

In the case that Capn12 is not secreted into the culture medium, the cells are then disrupted and Capn12 is obtained from the lysate using known protein isolation methods. The cells may be disrupted by high-frequency ultrasound, high pressure, as, for example, in a French press, by osmolysis, by the action of detergents, lytic enzymes or organic solvents, by homogenizers or by combination of two or more of the methods mentioned, as desired.

Capn12 purification can be achieved using known chromatographic methods such as molecular sieve chromatography (gel filtration), such as Q-Sepharose chromatography, ion exchange chromatography and hydrophobic chromatography, and also using other common methods such as ultrafiltration, crystallization, salting out, dialysis and native gel electrophoresis. Suitable methods are described, for example, in Cooper, F. G., Biochemische Arbeitsmethoden [The Tools of Biochemistry], Verlag Walter de Gruyter, Berlin, New York or in Scopes, R., Protein Purification, Springer Verlag, New York, Heidelberg, Berlin.

It is particularly advantageous to use for the isolation of the recombinant protein vector systems or oligonucleotides which elongate the cDNA by particular nucleotide sequences and thus code for modified polypeptides or fusion proteins which serve to simplify purification. Suitable modifications of this type are, for example, so-called tags which act as anchors, such as, for example, the modification known as the hexa-histidine anchor, or epitopes which can be recognized as antigens by antibodies (described, for example, in Harlow, E. and Lane, D., 1988, Antibodies: A Laboratory Manual. Cold Spring Harbor (N.Y.) Press). These anchors can be used to attach the proteins to a solid support such as, for example, a polymer matrix, which can, for example, be packed into a chromatography column, or to a microtiter plate or to another support.

At the same time, these anchors can also be used to recognize the proteins. It is also possible to use for recognition of the proteins common markers such as fluorescent dyes, enzyme markers which form a detectable reaction product after reaction with a substrate, or radioactive markers, alone or in combination with the anchors for derivatizing the proteins.

The invention also relates to the use of a Capn12 of the invention or a calpain protein of the invention as cysteine protease. Preference is given to the use in connection with natural substrates of Capn12, but it is also possible to use all substrates which bind to the active site of Capn12 and are cleaved there. Thus it is possible, for example, to use Capn12 as cysteine protease in molecular-biological and chemical methods.

Moreover, the invention relates to pharmaceutical compositions comprising a Capn12 of the invention, a calpain protein of the invention or a recombinant vector of the invention, and also at last one pharmaceutically suitable carrier or a diluent. The Capn12 of the invention, the calpain protein of the invention or the vector may be administered as such, preferably however together with a carrier or a diluent. Depending on the dosage form desired, said carrier may be present in solid or liquid form. Besides a Capn12 of the invention, a calpain protein of the invention or a recombinant vector of the invention, suitable pharmaceutical compositions may additionally contain further pharmaceutical active substances in a mixture or separated in a combination product. Such active substances can, for example, enhance the action of the contained Capn12, the calpain protein or the vector, they can have a different mode of action and thus have an additive effect or they can improve the overall constitution of the patient.

The invention furthermore relates to the use of a Capn12 of the invention, a calpain protein of the invention or a vector of the invention for preparing a medicament for treating disorders or pathological states which are connected with insufficient expression of Capn12. A treatment of the invention here includes the prevention of the development of the disease in a patient having an appropriate predisposition and the therapy of a disease already present by slowing down the progression or even by improving the state of the patient with the possibility of a complete cure.

In situations where there is a prevailing deficiency of Capn12 it is possible to use several methods for replacement. On the one hand, a Capn12 or a calpain protein of the invention may be administered directly or by gene therapy in the form of their coding nucleic acids (DNA or RNA) in a medicament of the invention. For administration as gene therapy, it is possible to utilize any vehicles, for example both viral (retroviral transfection) and non-viral vehicles (e.g. liposome transfection). Suitable vehicles may bind specifically to exactly defined target cells via suitable receptor molecules or the like and transform said target cells specifically. The transfection may take place inside the patient or removed cells are transfected in vitro and subsequently readministered to the patient. Suitable methods are described, for example, by Strauss and Barranger in Concepts in Gene Therapy (1997), Walter de Gruyter publisher. Stimulation of the endogenous gene represents another method for Capn12 replacement. It is also possible to block the turnover or the inactivation of Capn12 molecules of the invention, for example by proteases, in order to achieve an increase in the number of active Capn12 molecules. Finally, agonists of Capn12 may be employed in order to increase the activity of the Capn12 molecules present. At reduced Capn12 expression said method can only be a method for supporting the therapy.

Pharmaceutical compositions or medicaments of the invention may be present in the form of tablets, granules, powders, coated tablets, pastilles, pellets, capsules, suppositories, solutions, emulsions and suspensions for enteral and parenteral administration. Pharmaceutical compositions of the invention may preferably be contained in gels, lotions and creams for cutaneous application.

The particular dosage of the pharmaceutical compositions or medicaments of the invention and the particular dosage schedule are subject to the decision of the treating physician. The latter will select, depending on the chosen path of administration, on the activity of the particular medicament, on the nature and severity of the disorder to be treated, on the wellbeing of the patient and his response to the therapy, a suitable dose and a suitable dosage schedule. Thus, for example, the pharmacologically active substances can be administered to a mammal (human and animal) in doses of about 0.5 mg to 100 mg per kg of bodyweight per day. They can be administered in a single dose or in a plurality of doses.

The application areas include disorders and pathological states connected with insufficient Capn12 expression.

The invention also relates to the use of a Capn12 of the invention or a calpain protein of the invention for the screening for calpain protease effectors. Calpain protease effectors mean, for example, substances which can influence the activity of Capn12 and/or of other calpains, such as activators or inhibitors, or those substances which can act on the substrates of Capn12 during enzymatic catalysis, or Capn12-binding molecules such as immunoglobulins or low-molecular-weight Capn12-binding molecules, which can likewise modulate the biological function of Capn12. Capn12-binding molecules mean all natural and synthetic ligands and interaction partners of Capn12.

A suitable screening process comprises, for example, incubating Capn12 or a calpain protein of the invention with an analyte which contains an effector of a physiological or pathological Capn12 activity, for example cysteine protease activity, and determining the activity of Capn12, where appropriate by adding substrates and cosubstrates.

On the other hand, the following processes are based on the property of many effectors to bind to the target protein. Thus it is possible to immobilize Capn12 or the calpain protein of the invention, where appropriate after appropriate derivatization, on a support and to contact it with an analyte in which at least one Capn12 binding partner is suspected. The components of the analyte, which bind to the immobilized Capn12 or to the immobilized calpain protein of the invention, may then, where appropriate after an incubation phase, be eluted, determined and characterized. Accordingly however, it is also possible to immobilize the analyte and then test for binding of Capn12 molecules or of binding-capable Capn12 fragments to components of the analyte.

The invention furthermore relates to immunoglobulins which are specific for a Capn12 of the invention. Such immunoglobulins include monoclonal or polyclonal antibodies which can bind to characteristic Capn12 epitopes and also fragments thereof. Anti-Capn12 immunoglobulins are prepared in a manner familiar to the skilled worker. Immunoglobulins mean both polyclonal, monoclonal and, where appropriate, human or humanized antibodies or fragments thereof, single chain antibodies or else synthetic antibodies, and also antibody fragments such as Fv, Fab and F(ab′)₂. Suitable production methods are described, for example, in Campbell, A. M., Monoclonal Antibody Technology, (1987) Elsevier Verlag, Amsterdam, New York, Oxford and in Breitling, F. and Dübel, S., Rekombinante Antikörper (1997), Spektrum Akademischer Verlag, Heidelberg. In this way it is possible, for example starting from the amino acid sequences of the invention, to synthesize peptides which can be employed individually or in combination as antigens for the production of monoclonal or polyclonal antibodies.

The invention also relates to the use of immunoglobulins of the invention or polynucleotides of the invention for diagnosing disorders or pathological states connected with Capn12 expression. In this connection it is possible to determine the amount, activity and distribution of Capn12 or its underlying mRNA in the human organism. With the aid of immunoglobulins or Capn12-binding molecules it is possible, for example, to determine the Capn12 concentration in biological samples, e.g. cells or body fluids. With the aid of polynucleotides of the invention it is possible, for example, to evaluate the expression at mRNA level by means of the Northern blot technique or RT-PCR and, for example, to detect reduced expression and to diagnose a disorder connected thereto. It is also possible, with the aid of polynucleotides of the invention in the form of suitable probes, to detect gene defects or mutations with respect to the Capn12 gene and thus predisposition of a patient for particular disorders. From studying a large number of patients in clinical monitoring, it is furthermore possible to make statements about genetic causes and predispositions for particular disorders.

The nonlimiting examples below describe the invention in more detail with reference to the attached figures.

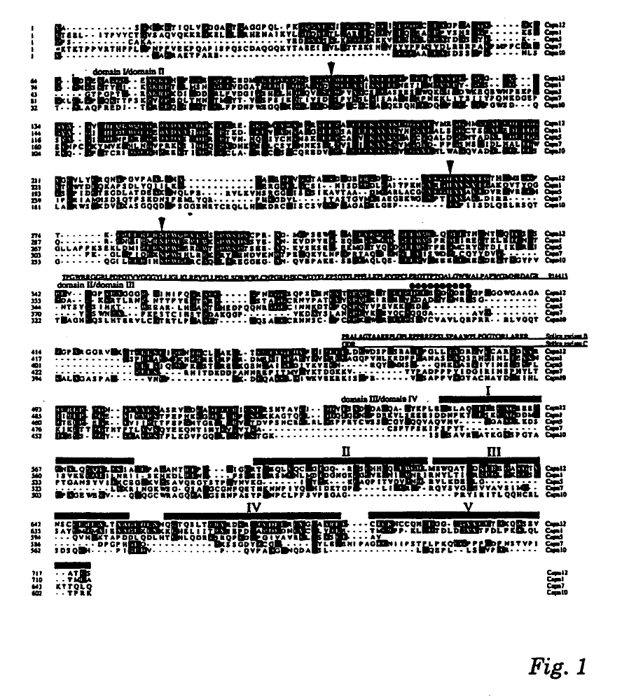

FIG. 1 shows a sequence comparison of the predicted Capn12 amino acid sequence with representative members of the vertebrate gene family of the large calpain subunit:

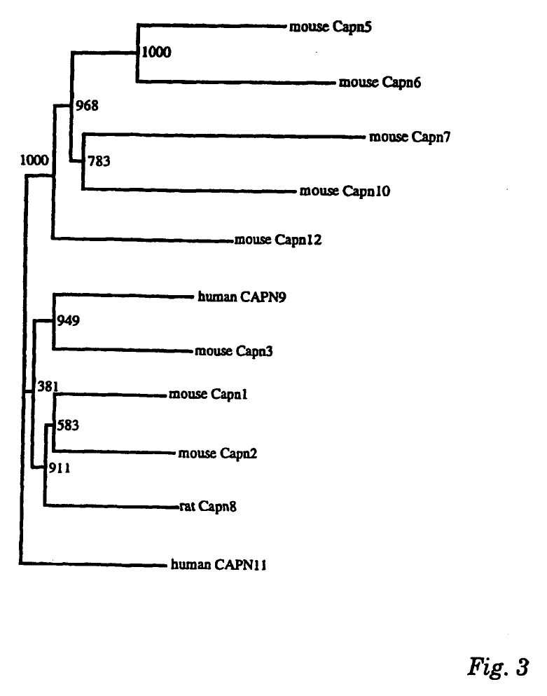

The predicted amino acid sequence (depicted here in one-letter code) of splice variant Capn12A was compared with members of the most important classes of the large calpain subunit which differ by various C-terminal domains. Capn1 has a conventional calmodulin-like C-terminal domain, while Capn5, Capn7 and Capn10 have C-terminal domains which are denoted N, T and X, respectively. Amino acids of other proteins, which are identical to those of Capn12, have a black background. Hyphens indicate gaps which have been introduced for alignment and thus for the best possible comparison of the sequences. The three conserved amino acids which are part of the active site of calpains are labeled by arrows. The calcium-binding EF-hand domains of Capn1 (Lin et al., 1997; Blanchard et al., 1997) are highlighted by a bar above the particular sequence and are numbered section by section. The calpain domains predicted from the crystal structure (Hosfield et al., 1999) are likewise indicated. To improve clarity, the first 122 amino acids of the predicted Capn7 protein which are found only in this protein are not shown and have been replaced by an “equal sign” (=). The distinctly acidic region in domain III, which can interact with calcium and possibly acts as an “electrostatic switch” of the protease activity, is indicated by circles above the relevant sequence. The C-terminal ends of the protein sequence predicted from 914413 cDNA and of the predicted protein sequences of splice variants B and C deviate from splice variant A and are shown from the point at which they differ from the protein sequence of splice variant Capn12A onward. The EMBL/GenBank accession numbers of the calpain sequences are given in the legend to FIG. 3.

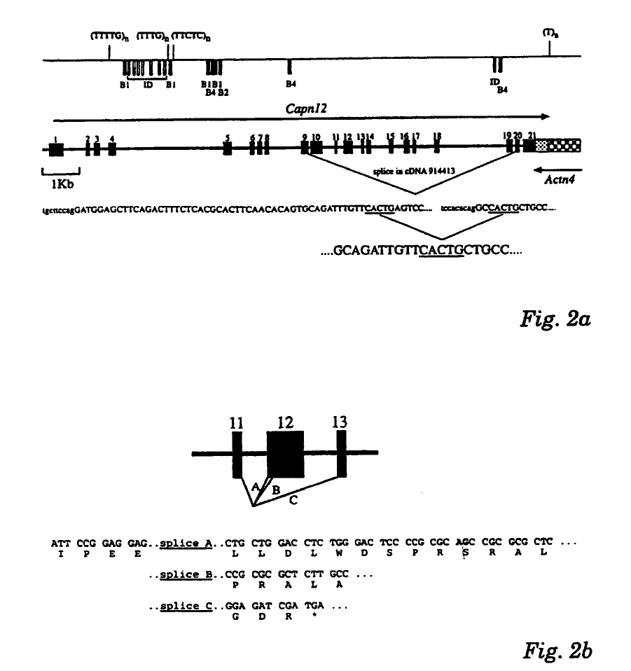

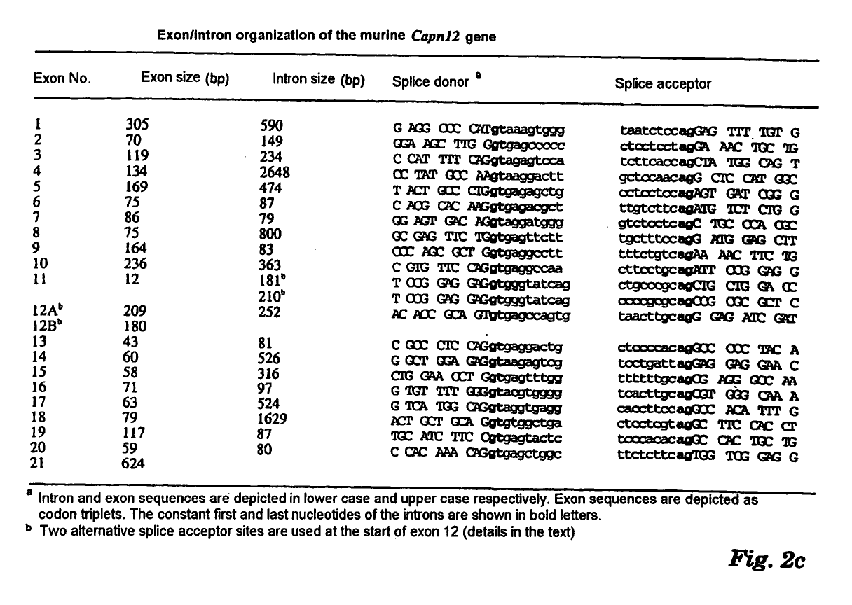

FIG. 2 shows the genomic structure of the Capn12 gene:

A. Diagrammatic representation of the intron/exon structure of the Capn12 gene. The black rectangles represent Capn12 exons. These are numbered consecutively. The checkered rectangle indicates the Actn4 exon which is located at the extreme 3′ end. The dotted rectangle indicates the exon sequence shared by Capn12 and Actn4. The arrows indicate the transcription direction of both genes. The position of the sequence repeats which were discovered in the sequence are indicated at the top. The splice event between exons 9 and 20, resulting in the mRNA transcript of clone 914413, and the part sequences surrounding the splice donor and splice acceptor sites of exons 9 and 20 of Capn12 are likewise indicated. Capital letters denote in each case the coding sequence and lower case letters the intron sequence. The sequence CACTG which is shared by the anomalous splice donor and splice acceptor sites and in which the anomalous splice event occurred is underlined. The adjoining 914413 cDNA sequence which connects said two exons with one another is likewise indicated.

B. A diagrammatric representation of exons 11, 12 and 13 shows the alternative splice variants A, B and C. The sequence of the shared exon 11 is shown on the left and the exon linked thereto which is used in the particular splice variant is shown on the right. The predicted amino acid sequence is indicated below the corresponding nucleotide sequence. The last two nucleotides of the splice acceptor in exon 12, AG, which are used in splice variant B, are depicted in bold.

C. The table shows the splice events of the individual exons together with the nucleotide sequence surrounding the particular splice donor and splice acceptor. Splice donor and splice acceptor are shown in bold. The size of the particular exons and introns is indicated.

FIG. 3 shows the phylogenetic tree of the mammalian gene family of the large calpain subunit:

The analysis was carried out with the aid of the program CLUSTAL, and the tree was produced using CLUSTREE. The particular length of the horizontal lines is proportional to the suspected phylogenetic distance; the vertical distances have no meaning. 1 000 bootstrap repeats were carried out and the values are indicated on the inside of the nodes. Preference was given to using murine sequences. Since these are not available for Capn8, Capn9 and Capn11, the orthologous sequences of rats and humans were used as an alternative. The EMBL/GenBank accession numbers of the sequences are: Capn1 (AF021847), Capn2 (Y10139), Capn3 (X92523), Capn5 (Y10656), Capn6 (Y12582), Capn7 (AJ012475), rat Capn8 (D14480), human CAPN9 (AF022799), Capn10 (AF126867) and human CAPN11 (AJ242832).

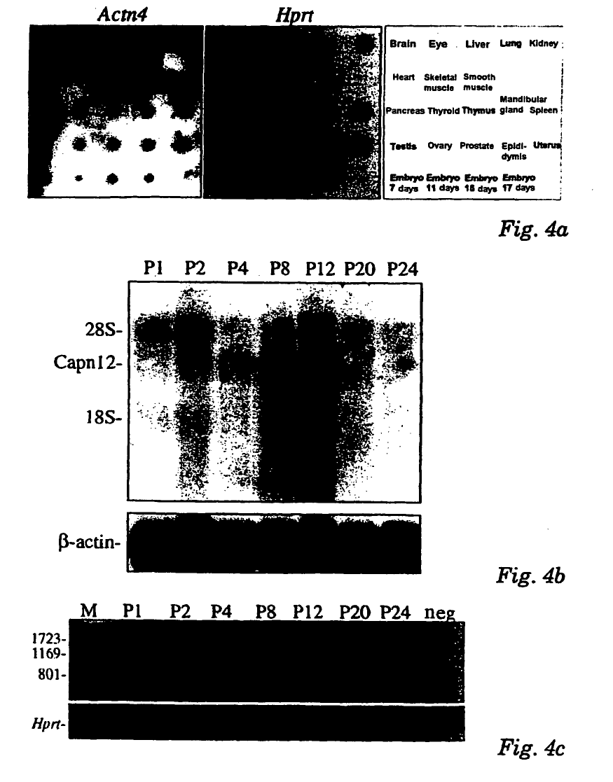

FIG. 4 shows mRNA expression analysis of Actn4 and Capn12:

A. Expression of Actn4 in various mouse tissues. A ³²P-labeled probe corresponding to the 3′ end of murine Actn4 CDNA was hybridized to a Clontech mouse Master Blot. The position of the RNAs on the filters is shown on the right. The blot was stripped and hybridized with a mouse Hprt probe (center) in order to check the amount of RNA bound. The exposure time was 48 hours.

B. A ³²P-labeled probe was hybridized to a Northern filter carrying RNAs from the skin of mice of the age indicated. To check the level of RNA applied, the blot was then hybridized once. more with a β-actin cDNA probe. The positions of the 28S and 18S rRNAs are marked and the specific Capn12 RNA band is labeled by an arrow. The exposure time was 144 hours for Capn12 and 2 hours for β-actin.

C. Capn12 RT-PCR of RNAs from the skin of mice of different postnatal age. M, pSM digested with HindIII, as molecular weight marker; the size of the bands is given in basepairs. Neg, negative control without using DNA. Sequencing of the highlighted PCR products confirmed that the amplified band corresponds to Capn12 cDNA.

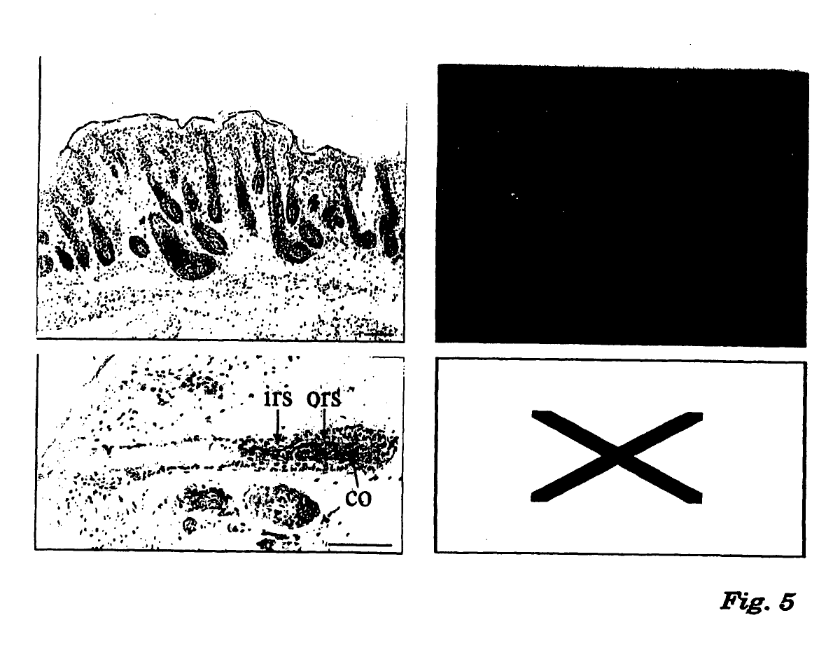

FIG. 5 shows an in situ hybridization on skin tissue sections from mouse embryos:

On the left, light-micrographs of the tissue sections are depicted. To the right thereof the corresponding image of the in situ hybridization is shown. Capn12 is selectively expressed in the cortex of the hair follicle (irs: inner root sheath; ors: outer root sheath; co: cortex).

### EXAMPLE 1

Screening of a Genomic Library

A cosmid library, constructed by cloning mouse 129/Sv DNA partially digested by Sau3A into the cosmid vector pSuperCos (Stratagene), was screened by PCR analysis using the Capn12-specific primers 5′-gaatggcgagtggcaacaggaag-3′ (SEQ ID NO: 9) and 5′-tggggctcagcacaaaactcat-3′ (SEQ ID NO: 10). The cosmid DNA was purified with the aid of the Qiagen plasmid Midi kit according to the manufacturer's instructions.

### EXAMPLE 2

cDNA Amplification by PCR

Five micrograms of total RNA were transcribed into CDNA using AMV reverse transcriptas and the Promega reverse transcription system. The PCRs were carried out in a 50 μl reaction volume containing 50 mM KCl, 10 mM Tris-HCl, pH 9, 0.1% Triton X-100, 2 units of Taq DNA polymerase, 50 pmol of both the forward and reverse primers and 0.1 ng of cDNA using a thermocycling protocol of 35 cycles comprising 15 s at 94° C., 30 s at 55° C. and 1 min at 72° C. The Capn12 forward and reverse primer sequences for the RT-PCR were 5′-ttcaagactttctcacg-3′ (SEQ ID NO: 11) and 5′-tcgcccccttgagtttattctga-3′ (SEQ ID NO: 12). The Hprt forward and reverse primer sequences were 5′-atgccgacccgcagtcccagcg-3′ (SEQ ID NO: 13) and 5′-ggctttgtatttggcttttcc-3′ (SEQ ID NO: 14).

### EXAMPLE 3

DNA Sequencing

A 20 μl reaction mixture containing 8 μl of BigDye reaction mix (Perkin-Elmer Biosystems), 500 ng of purified DNA and 10 pmol of primer was incubated over 30 cycles comprising 15 s at 94° C., 15 s at 50° C. and 2 min at 60° C. The reaction products were fractionated by polyacrylamide gel electrophoresis using an ABI 377 DNA sequencer and sequenced by dye terminator fluorescence with the aid of the Perkin-Elmer Biosystems sequence analysis software version 3.3. Further sequencing using synthesized oligonucleotides extended the DNA sequences. The sequences were assembled to make a contig with the aid of the SeqMan program of the DNASTAR series.

### EXAMPLE 4

Sequence Analyses

DNA sequences and amino acid sequences were studied regarding their homology with the nonredundant nucleotide, protein and EST databases of the National Center for Biotechnology Information (http://www.ncbi.nlm.nih.gov) with the aid of the BLAST program series (Altschul et al., 1990). The sequence comparison and alignment of amino acid sequences was carried out using CLUSTAL W (Thompson et al., 1994). Exon prediction was possible with the aid of the FGNENESH program which is available via the Sanger Centre Web Server (www.sanger.ac.uk). Repetitive sequences were identified using RepeatMasker (http://repeatmasker.genome.washington.edu). The phylogenetic analysis was carried out using the CLUSTREE program, available on the HUSAR server of the German Cancer Research Center, Heidelberg (www.dkfz-heidelberg.de).

### EXAMPLE 5

Northern Blot Hybridization

Total RNA from mouse tissues was isolated with the aid of the guanidine isothiocyanate method (Chomzynski and Sacchi, 1987). 10 μg of total RNA were fractionated by electrophoresis in a 1.4% (w/v) agarose gel containing, as described previously, 2.2 M formaldehyde (Sambrook et al., 1989), and blotted onto a Hybond-N nylon membrane (Amersham) according to the manufacturer's instructions. The blot was hybridized with a ³²P-labeled cDNA fragment corresponding to nucleotides 33-852 of the Capn12 cDNA sequence in Expresshyb hybridization solution (Clontech). The conditions of the hybridization and of the highly stringent washing were chosen according to the manufacturer's instructions. In a second step, the blot was hybridized with a cDNA probe of β-actins in order to check the amount of RNA bound.

### EXAMPLE 6

In Situ RNA Hybridization on Tissue Sections

Capn12 cDNA which was used as a template for the synthesis of RNAs, corresponding to nucleotides 33-852 of the sequence described, was cloned into the EcoRV site of pBluescript. Said cDNA fragment does not overlap with the Actn4 gene. ³³P-labeled sense and antisense RNAs were prepared by in vitro transcription of restriction enzyme-linearized plasmid DNA in a 12.5 μl reaction volume containing 1× transcription buffer (buffer for T7- and T3-RNA polymerases, from Strategene), 200 μM ATP, CTP, GTP, 40 μCi of α-³³P-UTP (Amersham), 10 mM DTT, 1 μg of linearized plasmid DNA, 40 units of RNAsin (Promega) and 10 units of RNA polymerase. After incubation at 37° C. for 2 hours, the template DNA was removed by adding 2 units of DNAaseI (Boehringer Mannheim) followed by incubation at 37° C. for 30 minutes. The reaction product was extracted, precipitated with ethanol and resuspended in 26 μl of DEPC-treated distilled water. The embryos were fixed in 4% (w/v) paraformaldehyde in PBS and 5-μm tissue sections obtained therefrom were transferred to precleaned SuperFrost Plus microscope slides (Menzel-Glaeser). Hybridization and washing conditions were as previously described (Dressler and Gruss, 1989). The hybridization temperature of choice was 55° C.

### EXAMPLE 7

Radiation Hybrid Mapping

The DNAs of the T31 radiation hybrid mapping panel (Research Genetics) were analyzed by PCR with the aid of two primer sets corresponding to Capn12 [set 1: 5′-gggagggccaggacaaggact-3′ (SEQ ID NO: 15), 5′-agggaaggctggaacaatggagaa-3′ (SEQ ID NO: 16), set 2: 5′-gaatggcgagtggcaacaggaag-3′ (SEQ ID NO: 17), 5′-ctggggctcagcacaaaactcat-3′ (SEQ ID NO: 18)] and to Capn5 (set 1: 5′-cggtgacactggactgggccttgc-3′ SEQ ID NO: 19), 5′-aagccgcctgcagagcactgtgg-3′ (SEQ ID NO: 20); set 2: 5′-cgggagtggacgggcccctg-3′ (SEQ ID NO: 21), 5′-ctcactttctgccattcctc-3′ (SEQ ID NO: 22)). The PCRs were carried out in a 20 μl reaction volume containing 50 mM KCl, 10 mM Tris-HCl, pH 9, 1.5 mM MgCl₂, 1 unit of Taq DNA polymerase, and 25 ng of DNA using a thermocycling protocol of 35 cycles comprising 15 s at 94° C., 30 s at 60° C. and 1 min at 72° C. The raw data were handed in for analysis to the mouse radiation hybrid database at Jackson Laboratory (www.jax.org/resources/documents/cmdata/rhmap/).

### EXAMPLE 8

Identification of Capn12

Novel calpain genes were identified by screening the publicly accessible EST databases were screened. Using the preliminary data obtained, a novel member of the mammalian gene family of the large calpain subunit which is characterized by a cell specific expression pattern was found and characterized.

The mouse EST database was screened using protein sequences of known vertebrate calpains and the TBLAST algorithm (Altschul et al., 1990). The translated protein of a 3′-EST, AA1314413, was typical for the family of the large calpain subunit. For this reason, the cDNA clone, 914413, corresponding to said EST clone was completely sequenced. The cDNA has a polyA tail and contains an open reading frame whose predicted protein shows homology to domains I and II of the large subunit of conventional calpains. However, the predicted sequence deviates from that of conventional calpains after domain II and ends shortly thereafter (FIG. 1).

Three observations indicate that said cDNA clone derives from an anomalous transcript. Firstly, the open reading frame has no homology to domains III or IV of other calpains, while all calpains identified so far have a typical four-domain structure. Secondly, it was impossible, using primers which had been constructed from the two ends of the obtained sequence, to amplify a transcript of this length from a large number of tissue cDNAs. Thirdly, human ESTs which are homologous to the 3′ end of AA1314413 and of which some showed homology to the calmodulin-like domain IV of calpains were identified. Therefore, the cDNA clone 914413 seems to be the result of an atypical or faulty RNA splicing event which has deleted the exons of said calpain gene, which code for domains III and IV.

In order to test this, a genomic DNA cosmid clone was isolated and sequenced. A continuous sequence (SEQ ID NO: 8) of 13116 bp in total was obtained. The gene prediction software (FGENESH) identified a potential gene having 21 exons and an exon/intron structure typical for the calpain gene family. The exons include those having a distinctive homology to domains III and IV of conventional calpains. To determine the exact exon/intron structure, mRNA isolated from the skin was analyzed by means of RT-PCR. The software predicted 20 of the 21 exons, allowing for some mistakes in the position of the donor splice site or the acceptor splice site. The intron/exon boundaries of the complete gene are shown in FIG. 2A and are summarized in Table 1. The mouse genome nomenclature committee named said gene Capn12. In the Capn12 intron sequence four simple sequence repeats and 16 SINES (short interspersed repeats; 4 B1, 1 B2, 3 B4 and 8 ID; FIG. 2A) were found. Compared with the mRNA sequence predicted from the genomic sequence, the cDNA clone 914413 seems to be the result of a faulty splice event because both the donor and the acceptor splice sites are atypical and most of the exons of domains III and IV are deleted thereby. Splicing takes place between exons 9 and 20 within a 5 base pair region, CACTG, which is shared by said two exons (FIG. 2A).

By means of RT-PCR, three alternative splice variants of Capn12 mRNA were identified (denoted Capn12A, Capn12B and Capn12C here). Splice variant A has an open reading frame which presumably encodes a protein of 720 amino acids (Mᵣ 80.5 kDa). The suggested starting methionine (cgaATGg) corresponds to the minimum consensus sequence of the translation start site (Kozak, 1996). Further 5′ start sites are excluded by a TAA stop codon which is located 39 nucleotides upstream in the reading frame from said ATG. The predicted amino acid sequence shows similarities to members of the family of the large calpain subunit and can be divided into the four domains I to IV typical for calpains (FIG. 1). Domain II of the Capn12 of the invention has the three amino acid residues (Cys105, His259 and Asn283) which are essential for the active sit of cysteine prot ases (Berti and Storer, 1995). Accordingly, the Capn12 of the invention, like most of the conventional calpains, has cysteine protease activity.

Each of the five Ca²⁺-binding sequences described for Capn2 (Blanchard et al., 1997; Lin et al., 1997) is to a certain extent conserved in the amino acid sequence of Capn12 (FIG. 1). The Capn2 crystal structure revealed an extremely acidic region in domain III, which could interact with Ca²⁺and act as an “electrostatic switch” of the protease activity (Strobl et al., 2000). The authors suspect that the large number of acidic residues in said region could reduce the Ca²⁺concentration necessary for activation. The corresponding region of Capn12 is likewise distinctly acidic (DEEEDDDDEE; FIG. 1). All in all, the primary amino acid sequence thus suggests that said protein has cysteine protease activity and binds calcium. A comparison of the predicted amino acid sequence with those of other vertebrate and invertebrate calpains shows a high sequence homology to Capn1 of humans and mice (39.9% and 39.75%, respectively).

The transcripts of splice variants A and B differ in the splice acceptor of exon 12, while exon 12 is missing entirely in variant C. The predicted proteins of the alternative splice variants B and C thereby show an amino acid sequence deviating in domain III and, owing to a reading frame shift, translation ends within said domain (FIG. 2B). Consequently, they presumably also lack the calmodulin-like, Ca²⁺-binding C-terminal domain. Analogously, it has been shown previously that Capn8 of rats and Capn5 of mice also form alternatively spliced transcripts coding for proteins which lack the C-terminal domain (Sorimachi et al., 1993; Dear et al., 1997). Surprisingly, the RT-PCR product of splice variant B was more abundant than that of splice variant A. Thus a Capn12 protein lacking a Ca²⁺-binding domain presumably represents a considerable part of the Capn12 protein pool. In contrast, the RT-PCR product of splice variant C was the least abundant.

### EXAMPLE 9

Phylogenetic Analysis of the Mammalian Large Calpain Subunit

The in each case complete amino acid sequences of representative members of all known mammalian large calpain subunits were subjected to a phylogenetic analysis. This made it possible to classify the calpains into three main groups (FIG. 3). The first group (A) is represented by Capn1, Capn2, Capn3, Capn8 and Capn9 and the second group (B) by Capn5, Capn6, Capn7, Capn10 and Capn12. Capn11, a highly deviating calpain (Dear et al., 1999), does not fit in any of the groups.

Group (A) contains all calpains having a calmodulin-like C-terminal domain, while group (B) contains all of those “atypical” calpains which presumably lack the ability of Ca²⁺binding. An exception is Capn12 which is generally more similar to group (B), except that it has a calmodulin-like C-terminal domain. Moreover, the phylogenetic analysis suggests that Capn12 is probably the oldest member of said group. Consequently, a predecessor of the Capn12 gene could be the originator of the genes of the atypical large calpain subunit, Capn12 having served as the source of both conventional and atypical proteins via alternative splicing.

### EXAMPLE 10

Chromosomal Localization

The location of the Capn12 gene on mouse chromosomes was determined by PCR analysis of the T31 radiation hybrid mapping panel with the aid of primers which bind within intron 1 of the Capn12 gene. The raw data were analyzed using the radiation hybrid map of the mouse genome (Van Etten et al., 1999) and the corresponding World Wide Web Server. The highest LOD score was 16, linked to marker D7Mit72. Other high LODs were 14.4, linked to D7Mit116, 14.4 linked to D7Mit77, and 13.9 linked to D7Mit267. Said markers were located on chromosome 7 at 9.4 (D7Mit77), 10.7 (D7Mit116), 10.4 (D7Mit72), and 11.0 cM (D7Mit267). The most logical sequence is: proximal-D7Mit77-D7Mit116-D7Mit72-Capn12-D7Mit267-distal. The region is orthologous to the human chromosome 19q13. The murine Capn5 gene has recently been located on mouse chromosome 7 likewise with the aid of a radiation hybrid mapping panel of the chromosomes of somatic mouse cells (Matena et al., 1998). In order to determine the exact distance between Capn5 and Capn12 on chromosome 7, the T31 panel was analyzed using mouse Capn5-specific PCR primers. The highest LOD value was 13.4 linked to D7Mit321. Other high LODs were 10.9, 8.5 and 6.9 linked to D7Mit184, D7Mit171 and D7Mit39, respectively. The most logical sequence of said locus is: proximal-D7Mit321-7cR-Capn5-42cR-D7Mit149-distal. The D7Mit321 marker was located at 48.5 cM on chromosome 7. Thus, Capn5 and Capn12 are syntenic, but are located on chromosome 7 at a marked distance from one another. Since the genes Actn4 and Capn12 overlap, as described below, Actn4 likewise has to be located on mouse chromosome 7. Since the human Actn4 gene was located on chromosome 19q13 (Kaplan et al., 2000), the human Capn12 ortholog is most probably likewise located in said region.

### EXAMPLE 11

Expression Analysis

A first in situ hybridization analysis on tissue sections from mouse embryos which had been carried out with the aid of 914413 cDNA for preparing strand-specific RNA probes led to confusing results, since the sense RNA, which was used as a control and which should hybridize to the antisense strand of Capn12, provided a hybridization signal in each experiment. A possible explanation for this phenomenon is that the antisense DNA strand likewise encodes an RNA. Studies of the DNA gene database identified over 200 ESTs corresponding to the 3′ end of the Capn12 gene. However, all ESTs with the exception of AA914413 correspond to the noncoding strand. The succession of overlapping ESTs formed a sequence having an open reading frame coding for the mouse ortholog of α-actinin-4 (Actn4). RT-PCR of various mouse tissue RNAs confirmed the sequence. The predicted mouse protein is 98.9% identical to the human Actn4. The last exon overlaps with the last exon of the Capn12 gene by 330 bp, but in opposite orientation (FIG. 2). It has recently been possible to show that mutations in the human Actn4 gene can cause familial focal segmental glomerulosclerosis (Kaplan et al., 2000).

RNA dot blot analysis and in situ hybridization using a specific probe showed that the Actn4 gene is expressed ubiquitously (FIG. 4). In contrast, it was impossible to obtain a hybridization signal with Capn12-specific cDNA probes in any of over 30 various tested poly(A+) RNA isolations from adult or embryonic tissue. Although the 914413 cDNA clone has been isolated from a mammary gland cDNA library, Northern blot and RT-PCR analysis in said tissue showed negligible expression levels. Only a more accurate RT-PCT analysis in connection with an in situ hybridization on mouse embryos of stages dE10,5 to dE18,5 and on various adult tissues showed that Capn12 is exclusively expressed in the skin. Here, Capn12 is expressed in the cortex of the hair follicle (FIG. 5).

Hair is subject to a cycle which lasts approximately 25 days in the mouse (Chase, 1965). The cycle is roughly divided into three phases: anagen (proliferation), catagen (regression) and telogen (rest phase). The dorsal skin of the adult mouse contains hair follicles of all hair cycle phases. In order to investigate in more detail in which phases of the cycle Capn12 mRNA is expressed, samples from the dorsal skin of mice were removed at various times after birth and the extracted RNAs were examined by Northern blot hybridization. The first hair cycle in mice is synchronized (Chase, 1965) and thus relatively pure hair follicle populations of a specific cycle phase can be examined. A Capn12 mRNA of approximately 3.5 kb can be detected in anagen (approximately P1-P16), but not in telogen (P19-P25) (FIG. 4B). The mRNA expression reaches its highest level approximately on day P12, half way through anagen. RT-PCR analysis of the same skin samples confirmed this result (FIG. 4C). Thus, Capn12 shows a highly specific mRNA expression pattern.

The gene family of the large calpain subunit can be classified based on various criteria, for example based on the protein structure, as mentioned above. Another classification criterion is the ubiquitous expression compared with the tissue-specific expression. Capn1, Capn2, Capn7 and Capn10 seems to be expressed ubiquitously, while the other calpains are characterized by tissue-specific expression of various extent. For example, Capn9 is expressed mainly in intestine and stomach, but is also detectable in other tissues (Li et al., 1998). In contrast, Capn11 is apparently exclusively expressed in particular cells of the testis (Dear and Boehm, 1999). Moreover, some calpain genes are expressed development-specifically. Capn5 is expressed, for example, in T cell precursors in the embryonic thymus, while expression in the thymus is downregulated postnatally (Dear and Boehm, 1999).

REFERENCES

Altschul, S. F., Gish, W., Miller, W., Myers, E. W. and Lipman, D. J. (1990). Basic local alignment sequencing tool. J. Mol. Biol. 215:403-410

Berti, P. J. and Storer, A. C. (1995). Alignment/Phylogeny of the papain superfamily of cysteine proteases. J. Mol. Biol. 246:273-283

Blanchard, H., Grochulski, P., Li, Y., Arthur, J. S. C., Davies, P. L., Elce, J. S. & Cygler, M. (1997). Structure of a Ca²⁺-binding domain reveals a novel EF-hand and Ca²⁺-induced conformational changes. Nature Struct. Biol. 4:532-538.

Braun, C., Engel, M., Theisinger, B., Welter, C. and Seifert, M. (1999). CAPN 8: isolation of a new mouse calpain-isoenzyme. Biochem. Biophys. Res. Commun. 260:671-675

Carafoli, E. and Molinari, M. (1998). Calpain: a protease in search of a function? Biochem. Biophys. Res. Commun. 247:193-203

Chan, S. L. and Mattson, M. P. (1999). Caspase and calpain substrates: roles in synaptic plasticity and cell death. J. Neurosci. Res. 58:167-190

Chase, H. B. (1965). Cycles and waves of hair growth. In: Lyne, A. B., Short, B. F. (Eds.). Biology of the Skin and Hair Growth. Angus and Robertson, Sydney, pp. 462-465

Chomczynski, P. and Sacchi, N. (1987). Single-step method of RNA isolation by acid guanidinium thiocyanate-phenol-chloroform extraction. Anal. Biochem. 162:156-159

Dear, T. N. and Boehm, T. (1999). Diverse mRNA expression patterns of the mouse calpain genes Capn5, Capn6 and Capn11 during development. Mech. Dev. 89:201-209

Dear, T. N., Matena, K., Vingron, M. and Boehm, T. (1997). A new subfamily of vertebrate calpains lacking a calmodulin-like domain: Implications for calpain regulation and evolution. Genomics 45:175-184

Dear, T. N., Moller, A. and Boehm, T. (1999). CAPN11: A calpain with high mRNA levels in testis and located on chromosome 6. Genomics 59:243-247

Dressler, G. R., Gruss, P., 1989. Anterior boundaries of Hox gene expression in mesoderm-derived structures correlate with the linear gene order along the chromosome. Differentiation 41, 193-201

Franz, T., Vingron, M., Boehm, T. and Dear, T. N. (1999). Capn7: A highly divergent vertebrate calpain with a novel C-terminal domain. Mamm. Genome 10:318-321

Hardman, M. J., Sisi, P., Banbury, D. N. and Byrne, C. (1998). Patterned acquisition of skin barrier function during development. Development 125, 1541-1552

Hosfield, C. M., Elce, J. S., Davies, P. L. and Jia, Z. (1999). Crystal structure of calpain reveals the structural basis for Ca²⁺-dependent protease activity and a novel mode of enzyme activation. EMBO J. 18:6880-6889

Kaplan, J. M., Kim, S., North, K. N., Rennke, I L, Correia, L., Tong, H. Q., Mathis, B. J., Rodfiguez-Perez, J. C., Allen, P. G., Beggs, A. H. and Pollak, M. R. (2000). Mutations in ACTN4, encoding alpha-actinin-4, cause familial focal segmental glomerulosclerosis. Nat. Genet. 24, 251-256

Kozak, M. (1996). Interpreting CDNA sequences: some insights from studies on translation. Mamin. Genome 7:563-574

Lee, H A., Sorimachi, H., Jeong, S-Y., Ishiura, S. and Suzuki, K. (1998). Molecular cloning and characterization of a novel tissue-specific calpain predominantly expressed in the digestive tract. Biol. Chem. 379, 175-183

Lin, G. D., Chattopadhyay, D., Maki, M., Wang, K. K., Carson, M., Jin, L., Yuen, P. W., Takano, E., Hatanaka, M., DeLucas, L. J. and Narayana, S. V. (1997). Crystal structure of calcium bound domain VI of calpain at 1.9 A resolution and its role in enzyme assembly, regulation, and inhibitor binding. Nat. Struct. Biol. 4:539-547

Matena, K., Boehm, T. and Dear, T. N. (1998). Genomic organization of mouse Capn5 and Capn6 genes confirms that they are a distinct calpain subfamily. Genomics 48:117-120

Mellgren, R. L. (1997). Evidence for participation of a calpain-like cysteine protease in cell cycle progression through late G1 phase. Biochem. Biophys. Res. Commun. 236:555-558

Richard, I., Broux, O., Allamand, V., Fougerousse, F., Chiannilkulchai, N., Bourg, N., Brenguier, L., Devaud, C., Pasturaud, P., Roudaut, C., Hillaire, D., Passos-Bueno, M., Zatz, M., Tischfield, J. A., Fardeau, M., Jackson, C. E., Cohen, D. and Beckmann, J. S. (1995). Mutations in the proteolytic enzyme calpain 3 cause Limb-Girdle Muscular Dystrophy Type 2A. Cell 81:27-40

Sambrook, J., Fritsch, E. F., Maniatis, T. 1989. Molecular Cloning. A Laboratory Manual. Cold Spring Harbor Laboratory Press, Cold Spring Harbor, pp. 7.43-7.45

Schoenwaelder, S. M., Yuan, Y., Cooray, P., Salem, H. H. and Jackson, S. P. (1997). Calpain cleavage of focal adhesion proteins regulates the cytoskeletal attachment of integrin alphaIIbbeta3 (platelet glycoprotein IIb/IIIa) and the cellular retraction of fibrin clots. J Biol. Chem. 272:1694-1702

Sorimachi, H., Ishiura, S. and Suzuki, K. (1993). A novel tissue-specific calpain species expressed predominantly in the stomach comprises two alternative splicing products with and without Ca²⁺-binding domain. J. Biol. Chem. 268:19476-19482

Sorimachi, H., Ishiura, S. and Suzuki, K. (1997). Structure and physiological function of calpains. Biochem. J. 328: 721-732.

Strobl, S., Fernandez-Catalan, C., Braun, M., Huber, R., Masumoto, H., Nakagawa, K., Irie, A, Sorimachi, H., Bourenkow, G., Bartunik, H., Suzuki, K. and Bode, W. (2000). The crystal structure of calcium-free human m-calpain suggests an electrostatic switch mechanism for activation by calcium. Proc. Natl. Acad. Sci. USA 97:588-592

Thompson, J. D., Higgins, D. G. and Gibson, T. J. (1994). CLUSTAL W: improving the sensitivity of progressive multiple sequence alignment through sequence weighting, position-specific gap penalties and weight matrix choice. Nucl. Acids Res. 22:4673-4680

Van Etten, W. J., Steen, R. G., Nguyen, H., Castle, A. B., Slonim, D. K., Ge, B., Nusbaum, C, Schuler, G. D., Lander, E S. and Hudson, T. J. (1999). Radiation hybrid map of the mouse genome. Nat. Genet. 22:384-387

Wang, K. K. (2000). Calpain and caspase: can you tell the difference? Trends Neurosci. 23:20-26

Wang, K. K. and Yuen, P. W. (1997). Development and therapeutic potential of calpain inhibitors. Adv. Pharmacol. 37:117-152

We claim:

1. A calpain protease 12 (Capn12), which comprises an amino acid sequence comprising the amino acids 1-342 of SEQ ID NO: 1, and functional equivalents thereof.

2. A Capn12 as claimed in claim 1, which has an amino acid sequence selected from SEQ ID NO: 1, SEQ ID NO: 2, SEQ ID NO: 3 or SEQ ID NO: 4, and functional equivalents thereof.

3. A Capn12 as claimed in claim 1 and 2, which has cysteine protease activity.

4. A calpain protein, which has at least one Capn12 as claimed in any of the preceding claims.

5. A polynucleotide, which codes for a Capn12 as claimed in any of claims 1 to 3, and functional equivalents thereof and polynucleotides hybridizable therewith or complementary thereto.

6. A polynucleotide as claimed in claim 5 having a nucleic acid sequence selected from SEQ ID NO: 5, SEQ ID NO: 6, SEQ ID NO: 7 or SEQ ID NO: 8.

7. An expression cassette comprising at least one regulatory nucleic acid sequence operatively linked with a polynucleotides as claimed in claim 5 or 6.

8. A recombinant vector, which carries a polynucletide as claimed in claim 5 or 6 or an expression cassette as claimed in claim 7.

9. A microorganism comprising at least one recombinant vector as claimed in claim 8.

10. A process for preparing a Capn12 as claimed in any of claims 1 to 3, wherein a microorganism which procedures Capn12 is cultured and the Capn12 is isolated from the culture.

11. The use of a Capn12 as claimed in any of claims 1 to 3 or of a calpain protein as claimed as claimed in claim 4 as cysteine protease.

12. A pharmaceutical composition comprising a Capn12 as claimed in any of claims 1 to 3, a calpain protein as claimed in claim 4 or a recombinant vector as claimed in claim 8.

13. The use of a Capn12 as claimed in any of claims 1 to 3, of a calpain protein as claimed in claim 4 or of a recombinant vector as claimed in claim 8 for preparing a medicament for treating disorders or pathological states connected with insufficient Capn12 expression.

14. The use of a Capn12 as claimed in any of claims 1 to 3 or of a calpain protein as claimed in claim 4 for screening for calpain protease effectors.

15. An immunoglobulin specific for a Capn12 as claimed in any of claims 1 to 3.

16. The use of immunoglobulins as claimed in claim 15 or of Capn12-binding molecules for diagnosing disorders or pathological states connected with a Capn12 expression.

17. The use of polynucleotides as claimed in claim 5 or 6 for diagnosing disorders or pathological states connected with a Capn12 expression.


SEQUENCE LISTING (22 sequences)

SEQ ID NO: 1 - protein, 720 aa, Mus sp.
MASGNRKVTIQLVDDGAGTGAGGPQLFKGQNYEAIRRACLDSGILFRDPCFPAGPDALGYDKLGPDSEKAKGVEWKRPHEFCAEPQFICEDMSRTDVCQGSLGNCWLLAAAASLTLYPRLLYRVVPPGQGFQDGYAGVFHFQLWQFGRWVDVVVDDKLPVREGKLMFVRSEQRNEFWAPLLEKAYAKLHGSYEVMRGGHMNEAFVDFTGGVGEVLYLRQNTPGVFAALRHALAKESLVGATALSDRGEIRTDEGLVKGHAYSVTGTHKMSLGFTKVRLLRLRNPWGRVEWSGPWSDSCPRWDMLPSEWRDALLVKKEDGEFWMELQDFLTHFNTVQICSLSPEVLGPSPAGGGWHIHIFQGRWVRGFNSGGSQPSAENFWTNPQFRLTLLEPDEEEDDDDEEGPWGGWGAAGARGPARGGRVPKCTVLLSLIQRNRRCLRAKGLTYLTVGFHVFQIPEELLDLWDSPRSRALLPGLLRADRSVFCARRDVSRRCRLPPGHYLVVPSASRVGDEADFTLRIFSERSHTAVEIDDVISADLDALQAPYKPLELELAQLFLELAGEEEELNALQLQTLISIALEPARANTRTPGEIGLRTCEQLVQCFGRGQRLSLHHFQELWGHLMSWQATFDKFDEDASGTMNSCELRLALTAAGFHLNNQLTQSLTSRYRDSRLRVDFERFVGCAARLTCIFRHCCQHLDGGEGVVCLTHKQWSEVATFS

SEQ ID NO: 2 - protein, 518 aa, Mus sp.
MASGNRKVTIQLVDDGAGTGAGGPQLFKGQNYEAIRRACLDSGILFRDPCFPAGPDALGYDKLGPDSEKAKGVEWKRPHEFCAEPQFICEDMSRTDVCQGSLGNCWLLAAAASLTLYPRLLYRVVPPGQGFQDGYAGVFHFQLWQFGRWVDVVVDDKLPVREGKLMFVRSEQRNEFWAPLLEKAYAKLHGSYEVMRGGHMNEAFVDFTGGVGEVLYLRQNTPGVFAALRHALAKESLVGATALSDRGEIRTDEGLVKGHAYSVTGTHKMSLGFTKVRLLRLRNPWGRVEWSGPWSDSCPRWDMLPSEWRDALLVKKEDGEFWMELQDFLTHFNTVQICSLSPEVLGPSPAGGGWHIHIFQGRWVRGFNSGGSQPSAENFWTNPQFRLTLLEPDEEEDDDDEEGPWGGWGAAGARGPARGGRVPKCTVLLSLIQRNRRCLRAKGLTYLTVGFHVFQIPEEPRALAGTAARRPLGFLRPPRREPSLSPAAWPLPGHICHAFDBCHAFLCHFGTQRLARRR

SEQ ID NO: 3 - protein, 462 aa, Mus sp.
MASGNRKVTIQLVDDGAGTGAGGPQLFKGQNYEAIRRACLDSGILFRDPCFPAGPDALGYDKLGPDSEKAKGVEWKRPHEFCAEPQFICEDMSRTDVCQGSLGNCWLLAAAASLTLYPRLLYRVVPPGQGFQDGYAGVFHFQLWQFGRWVDVVVDDKLPVREGKLMFVRSEQRNEFWAPLLEKAYAKLHGSYEVMRGGHMNEAFVDFTGGVGEVLYLRQNTPGVFAALRHALAKESLVGATALSDRGEIRTDEGLVKGHAYSVTGTHKMSLGFTKVRLLRLRNPWGRVEWSGPWSDSCPRWDMLPSEWRDALLVKKEDGEFWMELQDFLTHFNTVQICSLSPEVLGPSPAGGGWHIHIFQGRWVRGFNSGGSQPSAENFWTNPQFRLTLLEPDEEEDDDDEEGPWGGWGAAGARGPARGGRVPKCTVLLSLIQRNRRCLRAKGLTYLTVGFHVFQIPEEGDR

SEQ ID NO: 4 - protein, 447 aa, Mus sp.
MASGNRKVTIQLVDDGAGTGAGGPQLFKGQNYEAIRRACLDSGILFRDPCFPAGPDALGYDKLGPDSEKAKGVEWKRPHEFCAEPQFICEDMSRTDVCQGSLGNCWLLAAAASLTLYPRLLYRVVPPGQGFQDGYAGVFHFQLWQFGRWVDVVVDDKLPVREGKLMFVRSEQRNEFWAPLLEKAYAKLHGSYEVMRGGHMNEAFVDFTGGVGEVLYLRQNTPGVFAALRHALAKESLVGATALSDRGEIRTDEGLVKGHAYSVTGTHKMSLGFTKVRLLRLRNPWGRVEWSGPWSDSCPRWDMLPSEWRDALLVKKEDGEFWMELQDFLTHFNTVQICSLSPTPGWRRGGRLPDPQTVVGGGYLLIGLKLREVTLLPDSLQRWWLCNPGRPHKCWDYELEPSQTELPPFLLKPLHVSPCLERGTTPTQALGWWALPAPWGMNRDAGR

SEQ ID NO: 5 - DNA, 2498 bp, Mus sp.
ggagccacgccccccatgactcaggaggttaaagggcttgggtccatctgtgtgcccagagtgtccgaatggcgagtggcaacaggaaggtcaccatccagctggtggacgacggggccgggactggagctgggggcccacagctctttaaaggccagaactacgaagccatccgaagagcttgcctggattccgggatcctgtttcgtgacccttgctttcctgctggccctgatgcccttggctatgacaagctgggacctgactcagagaaggccaaaggggtggaatggaagaggccccatgagttttgtgctgagccccagttcatctgtgaagacatgagcagaacagatgtgtgccagggaagcttgggaaactgctggcttcttgcagctgctgcctccctcacactctaccccaggctcctgtaccgggtggtcccccctggacaaggtttccaagatggctacgcgggggtcttccattttcagctatggcagtttggccgctgggtggatgtggtggtagacgacaaactgcctgtgcgtgaggggaagctgatgttcgtgcgctcagaacaaaggaacgagttctgggcccctctgctggaaaaggcctatgccaagctccatggctcctacgaggtaatgcgaggaggtcacatgaacgaggcttttgtggactttacaggaggcgtgggtgaggttctctacttgagacaaaacactccaggtgtctttgctgcccttcgccacgcattggccaaggagtcccttgtgggtgctactgccctgagtgatcggggtgagatccgcacagatgaagggctggtgaagggacatgcttattctgtcacaggcacgcacaagatgtctctgggcttcaccaaggtgcggctgctgcggctgaggaacccctggggccgcgtggagtggtccgggccctggagtgacagctgcccacgctgggacatgctcccttctgagtggcgagatgccctgcttgtgaaaaaggaggatggcgagttctggatggagcttcaagactttctcacgcacttcaacacagtgcagatttgttcactgagtcctgaggtgttgggccccagccctgctggcggcggctggcatatccacatcttccagggccgctgggtgcgaggcttcaactccggtgggagtcagcccagcgctgaaaacttctggaccaacccccagttccggctgacactgctggagcctgatgaggaagaggatgacgatgatgaagagggaccctggggaggctggggagcggcaggggcccggggcccggcgagaggaggccgagtccccaagtgcacggtcctgttgtcactcatccagcgcaaccgccggtgtctgagggccaagggcctcacttacctcactgtgggcttccacgtgttccagattccggaggagctgctggacctctgggactccccgcgcagccgcgcgctcttgccgggactgctgcgcgccgaccgctcggttttctgcgcccgccgcgacgtgagccgtcgctgtcgcctgccgcctggccactacctggtggtacccagcgcctcgcgcgtaggcgatgaagccgacttcactctgcgcatcttctcggagcgcagccacaccgcagtggagatcgatgacgtgatcagcgcagacctggacgccctccaggccccctacaagcccctggagctggagttggcacagctatttttggagctggctggagaggaggaggaactcaacgctcttcagctgcagaccttaataagcattgctctggaacctgcgagggccaacaccaggacccctggagagattgggcttaggacctgcgaacagcttgtgcagtgttttgggcgtgggcaaagactgtccctacaccacttccaggagctctggggccatctcatgtcatggcaggccacatttgacaagtttgatgaagatgcctctgggacaatgaactcctgtgaactgaggctggcactgactgctgcaggcttccacctcaacaaccagctgacccagtccctcactagccgctaccgggacagccggctccgtgtggacttcgagcgcttcgtgggctgtgcagcccggctcacctgcatcttccgccactgctgccaacacctggatggcggcgagggggtcgtctgcctgacccacaaacagtggtcggaggtggctaccttctcataggtttgaagctgagggaggtcaccctgctgcccgactcactgtcacaaaggtggtggctatgtaaccctggccggcctcacaagtgctgggattacgagctggagccatcccaaacagaactgccacccttccttttgaagcctcttcatgtcagtccctgcttagagaggggcacaacccccacacaggcactgggctggtgggcactgccagctccttggggcatgaacagagatgcagggagaagatgacaccagagtccttctt

SEQ ID NO: 6 - DNA, 2469 bp, Mus sp.
ggagccacgccccccatgactcaggaggttaaagggcttgggtccatctgtgtgcccagagtgtccgaatggcgagtggcaacaggaaggtcaccatccagctggtggacgacggggccgggactggagctgggggcccacagctctttaaaggccagaactacgaagccatccgaagagcttgcctggattccgggatcctgtttcgtgacccttgctttcctgctggccctgatgcccttggctatgacaagctgggacctgactcagagaaggccaaaggggtggaatggaagaggccccatgagttttgtgctgagccccagttcatctgtgaagacatgagcagaacagatgtgtgccagggaagcttgggaaactgctggcttcttgcagctgctgcctccctcacactctaccccaggctcctgtaccgggtggtcccccctggacaaggtttccaagatggctacgcgggggtcttccattttcagctatggcagtttggccgctgggtggatgtggtggtagacgacaaactgcctgtgcgtgaggggaagctgatgttcgtgcgctcagaacaaaggaacgagttctgggcccctctgctggaaaaggcctatgccaagctccatggctcctacgaggtaatgcgaggaggtcacatgaacgaggcttttgtggactttacaggaggcgtgggtgaggttctctacttgagacaaaacactccaggtgtctttgctgcccttcgccacgcattggccaaggagtcccttgtgggtgctactgccctgagtgatcggggtgagatccgcacagatgaagggctggtgaagggacatgcttattctgtcacaggcacgcacaagatgtctctgggcttcaccaaggtgcggctgctgcggctgaggaacccctggggccgcgtggagtggtccgggccctggagtgacagctgcccacgctgggacatgctcccttctgagtggcgagatgccctgcttgtgaaaaaggaggatggcgagttctggatggagcttcaagactttctcacgcacttcaacacagtgcagatttgttcactgagtcctgaggtgttgggccccagccctgctggcggcggctggcatatccacatcttccagggccgctgggtgcgaggcttcaactccggtgggagtcagcccagcgctgaaaacttctggaccaacccccagttccggctgacactgctggagcctgatgaggaagaggatgacgatgatgaagagggaccctggggaggctggggagcggcaggggcccggggcccggcgagaggaggccgagtccccaagtgcacggtcctgttgtcactcatccagcgcaaccgccggtgtctgagggccaagggcctcacttacctcactgtgggcttccacgtgttccagattccggaggagccgcgcgctcttgccgggactgctgcgcgccgaccgctcggttttctgcgcccgccgcgacgtgagccgtcgctgtcgcctgccgcctggccactacctggtggtacccagcgcctcgcgcgtaggcgatgaagccgacttcactctgcgcatcttctcggagcgcagccacaccgcagtggagatcgatgacgtgatcagcgcagacctggacgccctccaggccccctacaagcccctggagctggagttggcacagctatttttggagctggctggagaggaggaggaactcaacgctcttcagctgcagaccttaataagcattgctctggaacctgcgagggccaacaccaggacccctggagagattgggcttaggacctgcgaacagcttgtgcagtgttttgggcgtgggcaaagactgtccctacaccacttccaggagctctggggccatctcatgtcatggcaggccacatttgacaagtttgatgaagatgcctctgggacaatgaactcctgtgaactgaggctggcactgactgctgcaggcttccacctcaacaaccagctgacccagtccctcactagccgctaccgggacagccggctccgtgtggacttcgagcgcttcgtgggctgtgcagcccggctcacctgcatcttccgccactgctgccaacacctggatggcggcgagggggtcgtctgcctgacccacaaacagtggtcggaggtggctaccttctcataggtttgaagctgagggaggtcaccctgctgcccgactcactgtcacaaaggtggtggctatgtaaccctggccggcctcacaagtgctgggattacgagctggagccatcccaaacagaactgccacccttccttttgaagcctcttcatgtcagtccctgcttagagaggggcacaacccccacacaggcactgggctggtgggcactgccagctccttggggcatgaacagagatgcagggagaagatgacaccagagtccttctt

SEQ ID NO: 7 - DNA, 2289 bp, Mus sp.
ggagccacgccccccatgactcaggaggttaaagggcttgggtccatctgtgtgcccagagtgtccgaatggcgagtggcaacaggaaggtcaccatccagctggtggacgacggggccgggactggagctgggggcccacagctctttaaaggccagaactacgaagccatccgaagagcttgcctggattccgggatcctgtttcgtgacccttgctttcctgctggccctgatgcccttggctatgacaagctgggacctgactcagagaaggccaaaggggtggaatggaagaggccccatgagttttgtgctgagccccagttcatctgtgaagacatgagcagaacagatgtgtgccagggaagcttgggaaactgctggcttcttgcagctgctgcctccctcacactctaccccaggctcctgtaccgggtggtcccccctggacaaggtttccaagatggctacgcgggggtcttccattttcagctatggcagtttggccgctgggtggatgtggtggtagacgacaaactgcctgtgcgtgaggggaagctgatgttcgtgcgctcagaacaaaggaacgagttctgggcccctctgctggaaaaggcctatgccaagctccatggctcctacgaggtaatgcgaggaggtcacatgaacgaggcttttgtggactttacaggaggcgtgggtgaggttctctacttgagacaaaacactccaggtgtctttgctgcccttcgccacgcattggccaaggagtcccttgtgggtgctactgccctgagtgatcggggtgagatccgcacagatgaagggctggtgaagggacatgcttattctgtcacaggcacgcacaagatgtctctgggcttcaccaaggtgcggctgctgcggctgaggaacccctggggccgcgtggagtggtccgggccctggagtgacagctgcccacgctgggacatgctcccttctgagtggcgagatgccctgcttgtgaaaaaggaggatggcgagttctggatggagcttcaagactttctcacgcacttcaacacagtgcagatttgttcactgagtcctgaggtgttgggccccagccctgctggcggcggctggcatatccacatcttccagggccgctgggtgcgaggcttcaactccggtgggagtcagcccagcgctgaaaacttctggaccaacccccagttccggctgacactgctggagcctgatgaggaagaggatgacgatgatgaagagggaccctggggaggctggggagcggcaggggcccggggcccggcgagaggaggccgagtccccaagtgcacggtcctgttgtcactcatccagcgcaaccgccggtgtctgagggccaagggcctcacttacctcactgtgggcttccacgtgttccagattccggaggagggagatcgatgacgtgatcagcgcagacctggacgccctccaggccccctacaagcccctggagctggagttggcacagctatttttggagctggctggagaggaggaggaactcaacgctcttcagctgcagaccttaataagcattgctctggaacctgcgagggccaacaccaggacccctggagagattgggcttaggacctgcgaacagcttgtgcagtgttttgggcgtgggcaaagactgtccctacaccacttccaggagctctggggccatctcatgtcatggcaggccacatttgacaagtttgatgaagatgcctctgggacaatgaactcctgtgaactgaggctggcactgactgctgcaggcttccacctcaacaaccagctgacccagtccctcactagccgctaccgggacagccggctccgtgtggacttcgagcgcttcgtgggctgtgcagcccggctcacctgcatcttccgccactgctgccaacacctggatggcggcgagggggtcgtctgcctgacccacaaacagtggtcggaggtggctaccttctcataggtttgaagctgagggaggtcaccctgctgcccgactcactgtcacaaaggtggtggctatgtaaccctggccggcctcacaagtgctgggattacgagctggagccatcccaaacagaactgccacccttccttttgaagcctcttcatgtcagtccctgcttagagaggggcacaacccccacacaggcactgggctggtgggcactgccagctccttggggcatgaacagagatgcagggagaagatgacaccagagtccttctt

SEQ ID NO: 8 - DNA, 13116 bp, Mus sp.
tggtcctcctaggcctgcccaccttttgtgtgctccaggtcattaagctgctaaactcgccacaactgagggctccgtgccccagggaggaaaccactgaagaagcgtccctgctccttcgcaccccaaaccatcaattaatattaacaagggagaatgctcctcgatgcctaaagacccccaacagggtacaaatggagcaggagccacgccccccatgactcaggaggttaaagggcttgggtccatctgtgtgcccagagtgtccgaatggcgagtggcaacaggaaggtcaccatccagctggtggacgacggggccgggactggagctgggggcccacagctctttaaaggccagaactacgaagccatccgaagagcttgcctggattccgggatcctgtttcgtgacccttgctttcctgctggccctgatgcccttggctatgacaagctgggacctgactcagagaaggccaaaggggtggaatggaagaggccccatgtaaagtggggctgggctgggacctgggtctgatgggggagggccaggacaaggactcctgggtctgagggaggaggaccaggtcctggactcttggatctgagggaggagggccagggcctgggtctgagggaggaggaccagggcctgaactcttgggtctgagggaggaggaccagggcctgaactcttgggtctgagggaggagggccagggcctgggtctgagggaggaggaccagggcctgaactcttgggtctgagggaggacgaccagggcctggactcttgggtctgagggaggagggccagagtcttagcctgagggatcagggccaggacatgaacccttgagtgtaagagagacaggctgaggtctagaatcctggttcttaggaaaagggagtgggggataagagcagactcagccacgggattcaaggggatccaggaaggcaaactcccacccacagactttcccaaggttggaggccctcactacctgggtactggtgtcagggctcaggcctctgacttctccattgttccagccttcccttacctggcttctctggaaccttaatcttccaggagttttgtgctgagccccagttcatctgtgaagacatgagcagaacagatgtgtgccagggaagcttgggtgagcccccttgtgactgtctggagcccctagacccaggacttgaacagctcctctcctctgtctcctgtccccatggcttctttcttcagtctgctggtctctggtccactcgtacctaatctgagcctctttcctcctcctcctaggaaactgctggcttcttgcagctgctgcctccctcacactctaccccaggctcctgtaccgggtggtcccccctggacaaggtttccaagatggctacgcgggggtcttccattttcaggtagagtccagttccttgctctgtgcctcaatttcccccgtggtagcatgatgacataggcttcacagttaccattatgtccctaccccagcgcaggaggactggaattccagaacttgggaagcagaaggcaaaagcgggggttggaggtaggaatcaggcagggtctggaagctgagccgctcctgccctgtgttttgttttgttttgttttgttttgtttttcttcaccagctatggcagtttggccgctgggtggatgtggtggtagacgacaaactgcctgtgcgtgaggggaagctgatgttcgtgcgctcagaacaaaggaacgagttctgggcccctctgctggaaaaggcctatgccaagtaaggactccgccccctcccaaagccccagccctcccagctgcagccccaagaacatgcccaagccacgtggagtactgacatcacatcgggggtcctccagacacccaacctaggaccctgaacccagtcatagcccgccatagccctagtatcatggcactctcctggaagaaccttcattttttggtattttattgagaaaagacctcatacaacctagcttgcccaggaattagctatgtagccaaatgagaccttgaactgagggttttgcctccatctcagaagtgctggggttccaggtgtgtgctaccaccccaggtttatgcggtgctgggtttgaacccagggtctcatgtatgcttggtaagccctctaccaactgagctacatccccaacctttatccattcagttattgtcttgttatgtaggccaggttggcctcaaactcataatcctccttcactgggcccttgtgtgcatagaatataggcatgcaccacaacccatggctaaagttaggaagggagtgtgtgtgagctggggatggaacccacgatctgtgcatgctgagccacatcccagctcctcactgggggattctaggcaggggctctaccactgagccacgcccccagctcctcactgggggattctaggcaggggctctaccactgagccacgcccccagcccctcactgggggattctaggcaggggctctaccactgagccacacccccagcccctcactgggggattctaggcaggggctctaccactgagccacgcccccagcccctcactgggggattctaggcaggggctctaccactgagccacgcccccagcccctcactgggggagtctaggcaggggctctaccactgagccacacccccagcccctcactaggggattctaggcaggggctctaccactgagccacgcccccagcccctcactgggggagtctaggcaggggctctaccactgagccacaccccaagcccctcactaggggattctaggcaggggctctaccactgagccacgcccccagcccctcactgggggattctaggcaggggctctaccactgagccatgcccccagcccctcactgggggattctaggcaggggctctaccactgagccacgcccccagccccttactgggggattctaggcaggggctctaccactgagccatgcccccagcccctcactgggggattctaggcaggggctctaccactgagccacgcccccagcccctcactgggggattctaggcaggggctctaccactgagccacgccccagcccctcactggggaattctaggcagaggctctaccactgaagcataaggttcagcctgtgaatcttctaatcttgtttgtttgcttgtttgtttgtttatttatggttcttcaagacaggatttcactgtgtaacttggctgtcctggaactcactctgtagagcaggctgacctcagactcatagagatctgcctgcttttgcctcctgagtgctgggattaaaggcatacaccactacccagcagaattttcaaatcttaaaggcctcctctcttctcttctcttctcttctcttctcttctcttctcttctcttctcttctcttctcttctctctttctttctttttttttttttgtggaaatgacattttccacaaacattctaagaatcccactgatactcatatttcccaaggatcctgaaatcccatcatctatcagaacctggatttgccaaatcttattccctcaagggcctttaacctcacgccatctctcatggtcctttgagacatcggcagcccatcctttatcataggattaggctaccgtgcgctggaaggcctgacaagtccccataggcatcgccttcacaggctcccagagcctcaaaggttgagggagagttgagaattctggtgcagctcttccatggcttccagactgcacagtttcatggaccctagagatgagaggcctagcatgtgtcagatgagtctcccacctcatctctgaatagttcagggattgagcctactcctattatcacagtagtactaagtgtactgaggcaggaggattgcaagtttgagggcagactgagatgcatagcaataccatgtctaaacaaaacacaaacacccaattagctgagcacttatagaacaactttgtctctagtactctgagagcagaggcaggtggatctctgggactctgagacaaatgtggtctacagagtgagttcttggtcagtcaaagcttggtctcaaagacaagagggagggctgggggctggggtggggaatctgtagagatggctcagtgttaagagcactggttgctcttccagaagacctgacttttattcccagtcacgactatgtgtaacttcagtaccagggatctgaggcttccatggacactgcacacatgacatgtggtgcacagacatacatgcaggcaaaacacatacatatagaaattacatacacatacacacaattggggaataggtcctggagacccttattctgatagagctcctgcccaagatgttctgtaccttagacctacttctacctgctccaacaggctccatggctcctacgaggtaatgcgaggaggtcacatgaacgaggcttttgtggactttacaggaggcgtgggtgaggttctctacttgagacaaaacactccaggtgtctttgctgcccttcgccacgcattggccaaggagtcccttgtgggtgctactgccctggtgagagctgggctcccatgtggacctccactagaccaacttagtcaaggatgaggtgggaggggagccttagcatccagtgtctttcttaccttctgcggttgactccccctctccccccagatcctcaatgatagattctaggccagagttctacatataagctacatccctccccccacctccatttttacttttcatttcgaggcaaagtctaagttacctacacgggccttgaacatgtcacgcatcagccttctgtgttgctcgaatcccaggcctgtagtgcagagtccgggtttcccccatctcctatctgtcactccaattgctctccccagctctctctctgagtcccttggcattttatgctgctttgagagctccgggattggaagcatgaggatgggttggggggctggggagagatgcttctacctcccacccgaggctcacaatcttcgcctcctccagagtgatcggggtgagatccgcacagatgaagggctggtgaagggacatgcttattctgtcacaggcacgcacaaggtgagacgctccataggtggactgggctaaccctaccctctgtaacgatgcccctcacaccaccctcactgatgactttgtcttcagatgtctctgggcttcaccaaggtgcggctgctgcggctgaggaacccctggggccgcgtggagtggtccgggccctggagtgacaggtaggatgggcttggggtgggtgggggcgtggtcaggggcgtggctccacatgtcttcctctcacattggtctcctcagctgcccacgctgggacatgctcccttctgagtggcgagatgccctgcttgtgaaaaaggaggatggcgagttctggtgagttcttagggacccactctaccggtgggaggtccgctgggacaggagccttagaacgcagggccagaaaggacacagagaaactcatgggatggatgggtcatgttgcagagcaatggtccctatcagctgtgatgtgggaatctaaatctatttttttgcaaagttagagcagaagcagtaagatcaggactataaagggcattgttttcagagggagaacactgaaattaggttagcttaaaactcactatatagaccaggctagtccttgtctcatggccatattttgacctcagctttccaaaaggcaaggatggaattacaggcatgaggggatctaaaggaatgtagagtcagtgattttgggagatttaattggaatagaaccatatttagggggaatctggggaggctttaactatatataatttaaaacttttctatttctccattggtggtgagaggatcagtcctctccttccactgtgagatgctaaggtcaaactctcagcttgtcaggcttggacagcagtggcttttattggctctgccattttcccagacctatttgcgggttttctaatgctaatttgaatatgttgagaggcgtttgtgactccttcccgagataaggtatttgtgaggacttggagacattgccaaggcctgaaggcttcggggtttctggagattggaagttattctgcagtctttagggaactgggggcacttctggggcccctcaagccgggctctggagtggctgggtacttttcacggctggtgctttccaggatggagcttcaagactttctcacgcacttcaacacagtgcagatttgttcactgagtcctgaggtgttgggccccagccctgctggcggcggctggcatatccacatcttccagggccgctgggtgcgaggcttcaactccggtgggagtcagcccagcgctggtgaggccttggggacccctgagaagcaaacttgggtgaggcttgtggcaggatgggaactccacctccttcttttctgtcagaaaacttctggaccaacccccagttccggctgacactgctggagcctgatgaggaagaggatgacgatgatgaagagggaccctggggaggctggggagcggcaggggcccggggcccggcgagaggaggccgagtccccaagtgcacggtcctgttgtcactcatccagcgcaaccgccggtgtctgagggccaagggcctcacttacctcactgtgggcttccacgtgttccaggtgaggccaaggtcaagttgagggtctggaggggcagagggtcacaagggcaccgttatgggcagaagtgtactgtgggttcaaagaggagtgccactgcagatatcattggagaaagggattcaggaacaggaagagaaaaacgttgagggtccgagagcaggaggggaccaaagggccagagaagggatgtgggcacaggtggaaaggaaagggttgggggaggggtcagagaggacctaggtcaaagatgaggaaatattaagggttcagaaagaaggaggggtgtgagaggtgtggaaggggaggaaggaaatctgcgagctctccaaccttcattcccttggtgttttcttcctgcagattccggaggaggtgggtatcagatgcggctccagaattaccctagggcttgatggaccagggcaggaagcctgggaacacgggagggcctggccagacagtctgggtgtgtgtgggaaatggcgcggtggaggctatcagagggttgggtggggagctcgggtgggtggtggtcatgcccccctgcccgcagctgctggacctctgggactccccgcgcagccgcgcgctcttgccgggactgctgcgcgccgaccgctcggttttctgcgcccgccgcgacgtgagccgtcgctgtcgcctgccgcctggccactacctggtggtacccagcgcctcgcgcgtaggcgatgaagccgacttcactctgcgcatcttctcggagcgcagccacaccgcagtgtgagccagtgtaccctccataagccttaccaggggcatcccgaccccggcccaggaacctcaatctagaatcataggccccgcccctggcaccaagccccgcccaggaatcacaaatccctgtccctgcatcttcagccctgccctacccagggattcccttctccccaaaacccacactgcctttgactatatccacttcctctgctgagacctccgcccgaacgcctcccctttttctgtaacttgcagggagatcgatgacgtgatcagcgcagacctggacgccctccaggtgaggactgttgtaggtggggacaagactctagagggcgggcagggctttgggaaggaactgaactcctcctccccacaggccccctacaagcccctggagctggagttggcacagctatttttggagctggctggagaggtaagagtcggggactggggatgcccagccaaatgacaacgagctcccctctctccttagatgtcttataaaacaaaacaaaccctaaaccaaatcaaacactgtagatcaggatatccaggaacagctatgtattctgtagcccagactggcctccttcgggttacccatgctggggttaaacctgagtcactttgctggggtttggggtatcttttctttattctgggaatgctcaaattgtctcaaggcctttgctgggtctgcacctccttcctctgaaggttcccatcccctgccagactcaaaacatctttcccaagtgccttcctctgtcacttgcccacggtgggcccccacagtgtgtctcccaccactgtcctgaccactctgaggacaggcctgcctcctctagctggaccctaggaaggcagccacagccatgccgtcagtcctatggagcacagggcctggcccagagtggattgttggctggatgttttgaagtgggttctttcctgattaggaggaggaactcaacgctcttcagctgcagaccttaataagcattgctctggaacctggtgagtttggctggaggttgaggtgggggtccttgcaactgaagcaccatagctatacaggctctatgtgtgatgaagctagggcgccaggcacaggaacaggacttcctacaaggttatgtgagggccatgatcactcgcagccacgccccacttcctctaagaggtgggggcagaaatgtagaaccccagcttggttggttcttcaggcatgaactctcagcacctgcttctatgatatgcccactgcagggagttagtctgcagtgctcttgcagtgttggcctacatgcaaggggtgctggatttttttgcagcgagggccaacaccaggacccctggagagattgggcttaggacctgcgaacagcttgtgcagtgttttggggtacgtggggtagtatatggagaggagggacagggatgctgggcttttccttgccttttaggggacattgattgtaaccaggtgtcctcacttgcagcgtgggcaaagactgtccctacaccacttccaggagctctggggccatctcatgtcatggcaggtaggtgagggttgagagcagctgcctccttctagacactgatattgtgtggatggacaaagggggcactgccaacgaggatataaagtccctgtcaccccatagtggccctctgagggcaccaaatgtagtgatctagagctgcctctggttcctgttggaattccaggtcccagctcagcttcttccttgccaggtgaccaaccacaggcctgtcacctccccttcgaggagcctctgcttagctactaatgggtactccttcaaggggaggagctcaagggtcccagaactgatcatagtgataactccctgctactgactcttccctaaccttcgtgggtagatggatttgaacttgtccccaacagcctgggagcttgtctccttctcacaggtgtagagtggtgcccacccagaagccaccagagctgaggccgtctcttagctacttcaaggtgcaagagcatcactctggggctggacttgtgatactgactcccacctgcctctccaccttccaggccacatttgacaagtttgatgaagatgcctctgggacaatgaactcctgtgaactgaggctggcactgactgctgcaggtgtggctgaggacctgggatgctgtagggacagcaacccatcctcaaattcttgtctgcatccctcagctgtggccatccctaataggctgtccacaagtgccagagcccatttccttccctggaggctctgactgcttatctgtggcatggctaatgtgtagtatggcaaggagcccacaagatgccacagaacaccccagataccctaaagcaccttatgaggctacggagttatacaacagaggatgaaaatcccatcctaagccatggagaaatgtatgttagggtgggattatcgtgatttcagaagaccgtcagctccatgcctccatgggttcatctgtgaccactaagtaggagccggggcaggcaggcagggggcggcacgtaggctagtgagaaatgagagactacaagtatgagacctagaatagtggccaagaacatggaagacaagatcccaaggcagagtccaaggtgggggccagggtgctgaactaaagcagtggacacaggacagaggggaggtcgggaacttactcgatcatccatccattcatcccagagtgcctggttgttttggataggagtctgataataatgtttgcctgggaatcttcagcaattctaagaggttgacagagggctcctgggtcaggaactactgccatctagccaggtttcccttcagccctgggccagcatagaccaatactcagggtacatggacatcagagggacaccgacctgcctcaggccacctagctctgggcatggtgtgcctggtgttcgtgggggtgggaggggcagcatctgttgaatgagcacacaaaggtacaatacaaacttgtacagttatctttgagactgtatggggctcatggaagctgggagggacaagtccttgggccttagggcttctagaaatccattgcattgtgattctacagcagatgtgacagagccaatgtctagactttaggtgcggcctcagaggaagagtcacacagtggtacccagtcggggagatagtccgtcaacctctgaaggcgcaatcacaaagctgcacctgttggcaccttgagaagcagcctaagcaacttaagtgtcacactaacttcccagagggctggggttgtagctcaacggagagagcatttgcttggcctatgcaagggccccggggttccacccccaacactccaaaacagccacaaaaggcccacatcagttggagagtgctcctcaagcgtgctggaggccctgagttctagatcgagtaccacataaaccacaggctgaactcttggcacccgaggaggggcaggggcctcaggagctggtgacagtccttggctatgtaggagttagaggacagcttctttcaaacagcacacaggaatgctgcgtaggtaaggaacttttacttgcaactccagtgtgagggccagagttcagatccccagcacccacgtgaagggcaagtgatctcggtgagcctcggcctcagtagagaaaggactgaggaagacgctccccatgtacgtgtgcccacccccaacactaaaataagcagcaccacacgtggatactgtaaacacaataaacaaggcggcctcctcgtaggcttccacctcaacaaccagctgacccagtccctcactagccgctaccgggacagccggctccgtgtggacttcgagcgcttcgtgggctgtgcagcccggctcacctgcatcttccgtgagtactcctggcaggcagggtagggtgtggtggggtgtgcatcagggctggtgctgcgtactcaccctggcctctcccacacaggccactgctgccaacacctggatggcggcgagggggtcgtctgcctgacccacaaacaggtgagctggcccgagggacagtgtggctctagcaccatcccagggcctctgcctcaagggtatctttcttttctcttcagtggtcggaggtggctaccttctcataggtttgaagctgagggaggtcaccctgctgcccgactcactgtcacaaaggtggtggctatgtaaccctggccggcctcacaagtgctgggattacgagctggagccatcccaaacagaactgccacccttccttttgaagcctcttcatgtcagtccctgcttagagaggggcacaacccccacacaggcactgggctggtgggcactgccagctccttggggcatgaacagagatgcagggagaagatgacaccagagtccttcttaaaaatattacatgttttattctcccatccccagagggtggtttatccagaaaccaagaaaataaaaatcaatcagaataaactcaagggggcgagtggagagaaacccattaacgaccaggcaggcaggccagcagcctgcctccacctcagaaggtccccagagacctctgcccaccgccacgaggggaaaatcaggagggactggggagggcattgaatcagctatgtcttcattatgagagtgagagaggtggcagagatatgcagctagatggatatatatttatataataaatccgtaagttaataaagtaaatagtaattctctggaaggtcttaagtttttaaagttttctttttttttttaagttttttttttcctttttttttttttaaatgatttttttgtttgtttctgttccattctttgtgttttgttggttttggtccttagaaaatctgagactcagaggccaggtgggctggggctgattgccccgcagccactcctgaggcagagaagggctatggcaggtcctctgctcctgggaggagccactggaatctggtccaggggagctgggtgccctctgctggacttcttagggcaggcggttcctggacaaggcacatggggctttggcctagatgtgagaggctttgaaggggcctcaggggcagaggggacctgggataggaaggtatctctggggcacaggagtccgttgtcccctccaatcggctaagaacccacagcacagcgtatatatttagcagaccagaaatgctgattgccaagcctccctcccctacaagactgagaaagagaggcctgcctagcccctccctgcctgaccccctagaaggaccacaaagagctctttgcatagatacagagtcagggtgggggcagggctcctcagcccctccgggaggccaagggagtctctgttcagggtggccaagggcctcacaggtcgctctccccatagagggctgtggagaaggacttgtagtcaagggcgccaggagcagcatcaggcccctggtagggtgccatgcgggcgatgcagtactcggcctggtcggggggcagctctctccgcagttcctcaacagtgatgaagttcta

SEQ ID NO: 9 - DNA, 23 bp, Artificial sequence
  Description of the artificial sequence Capn12-Primer
gaatggcgagtggcaacaggaag

SEQ ID NO: 10 - DNA, 22 bp, Artificial sequence
  Description of the artificial sequence Capn12-Primer
tggggctcagcacaaaactcat

SEQ ID NO: 11 - DNA, 17 bp, Artificial sequence
  Description of the artificial sequence Capn12-Primer
ttcaagactttctcacg

SEQ ID NO: 12 - DNA, 23 bp, Artificial sequence
  Description of the artificial sequence Capn12-Primer
tcgcccccttgagtttattctga

SEQ ID NO: 13 - DNA, 22 bp, Artificial sequence
  Description of the artificial sequence Capn12-Primer
atgccgacccgcagtcccagcg

SEQ ID NO: 14 - DNA, 21 bp, Artificial sequence
  Description of the artificial sequence Hprt-Primer
ggctttgtatttggcttttcc

SEQ ID NO: 15 - DNA, 21 bp, Artificial sequence
  Description of the artificial sequence Capn12-Primer
gggagggccaggacaaggact

SEQ ID NO: 16 - DNA, 24 bp, Artificial sequence
  Description of the artificial sequence Capn12-Primer
agggaaggctggaacaatggagaa

SEQ ID NO: 17 - DNA, 23 bp, Artificial sequence
  Description of the artificial sequence Capn12-Primer
gaatggcgagtggcaacaggaag

SEQ ID NO: 18 - DNA, 23 bp, Artificial sequence
  Description of the artificial sequence Capn12-Primer
ctggggctcagcacaaaactcat

SEQ ID NO: 19 - DNA, 24 bp, Artificial sequence
  Description of the artificial sequence Capn5-Primer
cggtgacactggactgggccttgc

SEQ ID NO: 20 - DNA, 23 bp, Artificial sequence
  Description of the artificial sequence Capn5-Primer
aagccgcctgcagagcactgtgg

SEQ ID NO: 21 - DNA, 21 bp, Artificial sequence
  Description of the artificial sequence Capn5-Primer
cgggagtggaacgggcccctg

SEQ ID NO: 22 - DNA, 20 bp, Artificial sequence
  Description of the artificial sequence Capn5-Primer
ctcactttctgccattcctc